(19) United States
(12) Patent Application Publication
(10) Pub. No.: US 2024/0153618 A1
(43) Pub. Date: May 9, 2024

(54) SYSTEMS AND METHODS FOR AUTOMATED COMMUNICATIONS

(71) Applicant: AVAIL MEDSYSTEMS, INC., Santa Clara, CA (US)

(72) Inventors: Daniel HAWKINS, Palo Alto, CA (US); Ravi KALLURI, San Jose, CA (US); Shivakumar MAHADEVAPPA, Fremont, CA (US)

(21) Appl. No.: 18/327,422

(22) Filed: Jun. 1, 2023

Related U.S. Application Data
(63) Continuation of application No. PCT/US21/46732, filed on Aug. 19, 2021.
(60) Provisional application No. 63/121,826, filed on Dec. 4, 2020.

(51) Int. Cl. G16H 40/20; A61B 90/96; G06K 7/10; H04L 67/12; H04L 67/141

(52) U.S. Cl. CPC G16H 40/20; A61B 90/96; H04L 67/141; G06K 7/10861; H04L 67/12

(57) ABSTRACT

Systems and methods for medical resource intelligence are provided. Systems and methods are provided for identifying appropriate remote users to contact based on a plurality of factors including availability and operator preference.

### CROSS-REFERENCE

This application is a continuation of International Patent Application PCT/US21/46732, filed Aug. 19, 2021, which claims priority to U.S. Provisional Patent Application No. 63/121,826, filed Dec. 4, 2020, each of which is incorporated herein by reference in its entirety.

### BACKGROUND OF THE INVENTION

Medical products may be used during medical procedures and may be associated with vendors that provide the medical products. Traditionally, vendor representatives may be present in-person during the medical procedures where associated products are being used, to provide support. Such in-person support can be time consuming and costly, as it requires that the vendor representative physically make his or her way to the various locations of the related medical procedures.

Traditionally, preferences for individual medical personnel for various procedures may be known. For example, surgeons may have surgeon preference cards that list the preferred medical products for various procedures. However, these preference cards may become out of date unless surgeons actively update them and may not actually reflect the products that are used during a procedure. Coordinating and initiating communications during medical procedures such as surgery can be a distraction. There is a need for an automated communication system that can recognize the medical devices being used and coordinate that information with a preferred list of remote users based on the operator's preference, and facilitate the initiating of communications with the remote users through a hands-free means, such as voice recognition.

### SUMMARY OF THE INVENTION

A need exists for improved systems and methods for pre-medical procedures. A need exists for systems and methods that allow for identifying appropriate remote users to contact based on the products that are being used during a procedure.

Aspects of the invention are directed to a method of identifying medical product usage during a medical procedure, said method comprising: scanning via a camera a graphical code on a medical product; recognizing, with aid of one or more processors, the one or more medical products based on graphical code; identifying, with aid of one or more processors, a procedure associated with the one or more medical products and a remote user to contact; and communicating to an operator whether the remote user is available. Identifying can comprise identifying the remote user based on a preference list or based on a plurality of factors including availability and experience. Graphical codes can be QR codes, barcodes (e.g., 1D, 2D, 3D barcodes), symbols, letters, numbers, characters, shapes, sequences of lights or images, icons, or any other graphical code that may be useful for identifying the medical product.

Disclosed herein is a method of tracking medical product usage during a medical procedure, said method comprising recognizing, with aid of one or more sensors, one or more medical products; identifying, with aid of the one or more sensors, a procedure associated with the one or more medical products and a remote user to contact; and communicating to an operator whether the remote user is available. Recognizing can comprise scanning one or more graphical codes on the one or more medical products. The one or more graphical codes can comprise a barcode or QR code. Identifying the first remote user can be based on a preference list. Identifying the remote user can be based on a plurality of factors including availability, experience, location of the remote user, location of the operator, or identity of a healthcare facility.

The method can comprise identifying a second remote user when the remote user is not available. The second remote user can be identified based on a preference list of the operator. The second remote user can be specified by the operator. The method can comprise accessing a schedule of the remote user. The schedule of the remote user can be automatically updated. The method can further comprise, before (a), recognizing the operator. The operator can be recognized via voice recognition, password login, facial identification, or any combination thereof.

The method can comprise monitoring the length of time the operator is using the one or more medical products. The method can comprise determining an amount of time the one or more medical products is expected to be used based on the identified procedure. The method can comprise, upon recognizing that the length of time the operator is using the one or more medical products is greater than the amount of the one or more medical products is expected to be used based on the identified procedure, prompting the operator with an option to contact the first remote user. Commands from the operator can be received via a touchless interface. The touchless interface can comprise voice control or live video.

Disclosed herein is a method of tracking medical product usage during a medical procedure, said method comprising: recognizing, with aid of one or more sensors, one or more medical products; identifying, with aid of the one or more processors, a procedure associated with the one or more medical products and a remote user to contact; and automatically connecting the remote user to an operator. The method can comprise monitoring the length of time the operator is using the one or more medical products. The method can comprise determining an amount of time the one or more medical products is expected to be used based on the identified procedure. The method can comprise, upon recognizing that the length of time the operator is using the one or more medical products is greater than the amount of the one or more medical products is expected to be used based on the identified procedure, automatically contacting the remote user. Identifying the remote user can be based on a preference list.

Additional aspects and advantages of the present disclosure will become readily apparent to those skilled in this art from the following detailed description, wherein only exemplary embodiments of the present disclosure are shown and described, simply by way of illustration of the best mode contemplated for carrying out the present disclosure. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an example of a video capture system, in accordance with embodiments of the invention.

FIG. 2 shows an example of a medical resource intelligence system 220 configured to communicate with various devices, in accordance with embodiments of the invention.

FIG. 3A shows an example of medical console identifiers, in accordance with embodiments of the invention.

FIG. 3B shows an example of how medical console identifiers may affection functionality of medical consoles, in accordance with embodiments of the invention.

FIG. 4 shows an example of how practitioner preference cards may be accessed, in accordance with embodiments of the invention.

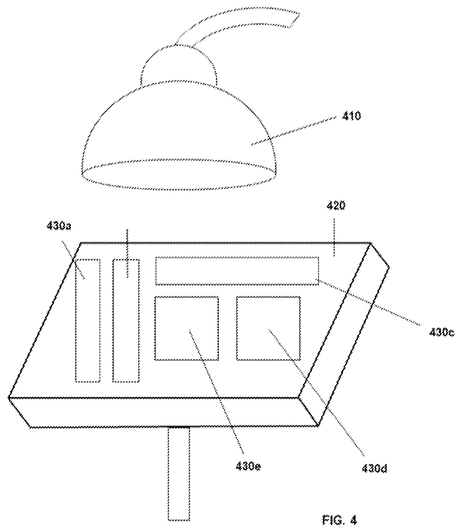

FIG. 5 shows a illustration of how scheduling may be updated in real-time, in accordance with embodiments of the invention.

FIG. 6 shows an example of medical products that may be recognized using a video capture system, in accordance with embodiments of the invention.

FIG. 7 shows an example of one or more factors that may be assessed in determining a remote user to contact, in accordance with embodiments of the invention.

FIG. 8 shows an example of a user interface displaying medical resource intelligence to a vendor representative, in accordance with embodiments of the invention.

FIG. 9 shows an exemplary computer system, in accordance with embodiments of the invention.

FIG. 10 shows a flow chart of an example of how an individual may use a console as described herein.

FIG. 11 shows a flow chart of an example of how the console can be used to monitor a procedure

### DETAILED DESCRIPTION OF THE INVENTION

While preferable embodiments of the invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

The invention provides systems and methods for preparing for medical procedures. Various aspects of the invention described herein may be applied to any of the particular applications set forth below. The invention may be applied as a part of a health care system or communication system. It shall be understood that different aspects of the invention can be appreciated individually, collectively or in combination with each other.

The systems and methods provided herein may be useful for assessing appropriate remote users to contact for a medical procedure. One or more factors based on collected data may be useful for identifying a remote user to contact. Performance of one more individuals involved in a medical procedure may be assessed. For example, performance of a remote user, such as a vendor representative, may be assessed. This may advantageously improve a selection of remote users to support a procedure in the future.

Systems and methods are provided for preparation for medical procedures. In some instances, a procedure preparation system may be able to access historical and real-time information in order to provide outputs that may aid in preparation before a procedure. For example, practitioner preferences may be accessible and used to prepare tools or individuals associated with the medical procedure. In some instances, a procedure preparation system may be able to provide information to a medical practitioner about a procedure to be performed, and associated steps. Practitioner preferences may include remote users that provide support during medical procedures (e.g. vendor representatives or other practitioners, consultants, and/or specialists).

Furthermore, systems and methods for accessing the procedure preparation system may be provided. For example, an identifier may be used to log into a medical console that may access one or more medical resource. The login identifier may include a location-based component and a practitioner-based component, which may affect the information that the medical console is able to access. The login identifier may affect the data that may be accessed in order to prepare for a medical procedure.

The systems and methods provided herein may utilize a video capture system in order to capture images during the medical procedure.

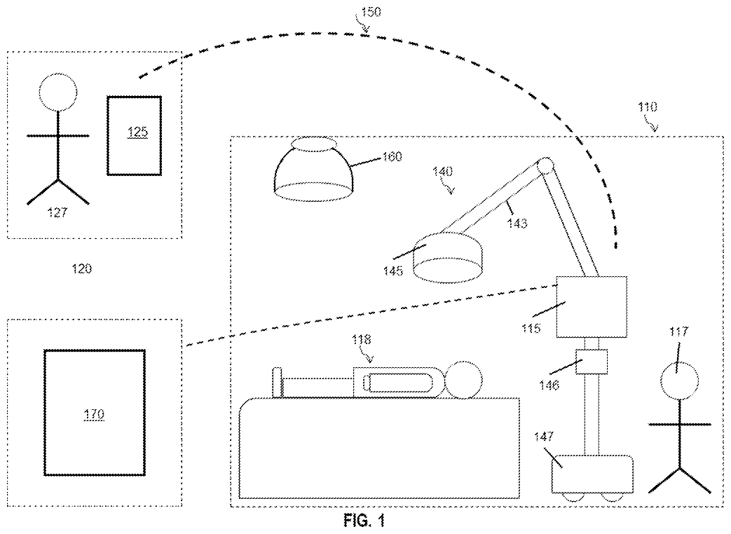

FIG. 1 shows an example of a video capture system utilized within a medical suite, such as an operating room. The video capture system may optionally allow for communications between the medical suite and one or more remote individuals, in accordance with embodiments of the invention. Communication may optionally be provided between a first location 110 and a second location 120.

The first location 110 may be a medical suite, such as an operating room of a health care facility. A medical suite may be within a clinic room or any other portion of a health care facility. A health care facility may be any type of facility or organization that may provide some level of health care or assistance. In some examples, health care facilities may include hospitals, clinics, urgent care facilities, out-patient facilities, ambulatory surgical centers, nursing homes, hospice care, home care, rehabilitation centers, laboratory, imaging center, veterinary clinics, or any other types of facility that may provide care or assistance. A health care facility may or may not be provided primarily for short term care, or for long-term care. A health care facility may be open at all days and times, or may have limited hours during which it is open. A health care facility may or may not include specialized equipment to help deliver care. Care may be provided to individuals with chronic or acute conditions. A health care facility may employ the use of one or more health care providers (a.k.a. medical personnel/medical practitioner). Any description herein of a health care facility may refer to a hospital or any other type of health care facility, and vice versa.

The first location may be any room or region within a health care facility. For example, the first location may be an operating room, surgical suite, clinic room, triage center, emergency room, or any other location. The first location may be within a region of a room or an entirety of a room. The first location may be any location where an operation may occur, where surgery may take place, where a medical procedure may occur, and/or where a medical product is used. In one example, the first location may be an operating room with a patient 118 that is being operated on, and one or more medical personnel 117, such as a surgeon or surgical assistant that is performing the operation, or aiding in performing the operation. Medical personnel may include any individuals who are performing the medical procedure or aiding in performing the medical procedure. Medical personnel may include individuals who provide support for the medical procedure. For example, the medical personnel may include a surgeon performing a surgery, a nurse, an anesthesiologist, and so forth. Examples of medical personnel may include physicians (e.g., surgeons, anesthesiologists, radiologists, internists, residents, oncologists, hematologists, cardiologists, etc.), nurses (e.g., CNRA, operating room nurse, circulating nurse), physicians' assistants, surgical techs, and so forth. Medical personnel may include individuals who are present for the medical procedure and authorized to be present.

Medical resources may include medical products, medical personnel, locations, instruments, utilities, or any other resource that may be involved for a medical procedure.

Medical products may include devices that are used alone or in combination with other devices for therapeutic or diagnostic purposes. Medical products may be medical devices. Medical products may include any products that are used during an operation to perform the operation or facilitate the performance of the operation. Medical products may include tools, instruments, implants, prostheses, disposables, or any other apparatus, appliance, software, or materials that may be intended by the manufacturer to be used for human beings. Medical products may be used for diagnosis, monitoring, treatment, alleviation, or compensation for an injury or handicap. Medical products may be used for diagnosis, prevention, monitoring, treatment, or alleviation of disease. In some instances, medical products may be used for investigation, replacement, or modification of anatomy or of a physiological process. Some examples of medical products may range from surgical instruments (e.g., handheld or robotic), catheters, endoscopes, stents, pacemakers, artificial joints, spine stabilizers, disposable gloves, gauze, IV fluids, drugs, and so forth.

Medical personnel may be considered as medical resources as well. For example, the number and types of individuals that may be required to be present at a medical procedure may be considered as a medical resource.

A video capture system may have one or more cameras. The video capture system may also comprise a local communication device 115. The local communication device may optionally communicate with a remote communication device 125.

One or more cameras may be integral to the communication device. Alternatively, the one or more cameras may be removable and/or connectable to the communication device. The one or more cameras may face a user when the user looks at a display of the communication device. The one or more cameras may face away from a user when the user looks at a display of the communication device. In some instances, multiple cameras may be provided which may face in different directions. The cameras may be capable of capturing images at a desired resolution. For instance, the cameras may be capable of capturing images at least a 6 mega pixel, 8 mega pixel, 10 mega pixel, 12 mega pixel, 20 mega pixel, 30 megapixels, 40 megapixels, or any number of pixels. The cameras may be capable of capturing SD, HD, Full HD, WUXGA, 2K, UHD, 4K, 8K, or any other level of resolution. A camera on a rep communication device may capture an image of a vendor representative. A camera on a local communication device may capture an image of a medical personnel. A camera on a local communication device may capture an image of a surgical site and/or graphical codes on medical tools, instruments or products.

The communication device may comprise one or more microphones or speakers. A microphone may capture audible sounds such as the voice of a user. The communication device may be configured to recognize a practitioner's voice and provide a menu of options which the practitioner can select by speaking. The interface can be touchless. A microphone array in the console may capture audio/speech in the room. For instance, the rep communication device microphone may capture the speech of the vendor representative and a local communication device microphone may capture the speech of a medical personnel. One or more speakers may be provided to play sound. For instance, a speaker on a rep communication device may allow a vendor representative to hear sounds captured by a local communication device, and vice versa.

In some embodiments, an audio enhancement module may be provided. The audio enhancement module may be supported by a video capture system. The audio enhancement module may comprise an array of microphones that may be configured to clearly capture voices within a noisy room while minimizing or reducing background noise. The audio enhancement module may be separable or may be integral to the video capture system. The video capture system can be used to automatically identify graphical codes on medical equipment and products in the vicinity of the console. The console can associate the medical equipment and products with the preferences of the practitioner (e.g. using a practitioner's preference card as described herein).

A medical console may comprise a communication device. The communication device may be an integral part of the medical console or may be a separate part of the medical console. A communication device may comprise a display screen. The display screen may be a touchscreen. The display screen may accept inputs by a user's touch, such as finger. The display screen may accept inputs by a stylus or other tool.

A communication device may be any type of device capable of communication. For instance, a communication device may be a smartphone, tablet, laptop, desktop, server, personal digital assistant, wearable (e.g., smartwatch, glasses, etc.), or any other type of device.

In some embodiments, a local communication device 115 may be supported by a medical console 140. The local communication device may be permanently attached to the medical console or may be removable from the medical console. Optionally, a local communication device may be an integral part of the medical console. In some instances, the local communication device may remain functional while removed from the medical console. The medical console may optionally provide power to the local communication device when the local communication device is attached to (e.g., docked with) the medical console. The medical console may be mobile console that may move from location to location. For instance, the medical console may include wheels that may allow the medical console to be wheeled from location to location. The wheels may be locked into place at desired locations. The medical console may optionally comprise a lower rack and/or support base 147. The lower rack and/or support base may house one or more components, such as communication components, power components, auxiliary inputs, and/or processors. A medical console may comprise one or more input ports to accept inputs from one or more auxiliary devices. For example, one or more inputs for endoscopy, fluoroscopy, ECG, etc. may be provided to the medical console. The information from one or more auxiliary devices may be displayed on a display of a medical console and/or remote user.

The medical console may optionally include one or more sensors (e.g., cameras 145, 146, scanners, etc.). The sensors (e.g., cameras) may be capable of capturing images of the patient 118, or portion of the patient (e.g., surgical site). The sensors (e.g., cameras or scanners) may be capable of capturing images of graphical codes on the medical devices. The sensor(s) (e.g., cameras) may be capable of capturing images of the graphical codes of the medical devices as they rest on a tray, or when they are handled by a medical personnel and/or used at the surgical site. The sensor(s) (e.g., cameras or scanners) may be capable of capturing images of graphical codes at any resolution, such as those described elsewhere herein. The scanner(s) (e.g., cameras) may be used to capture a still images and/or video images. The sensor(s) (e.g., cameras or scanners) may be capturing images in real time.

One or more of the sensor(s) (e.g., cameras) may be movable relative to the medical console. For instance, one or more sensor(s) (e.g., cameras) may be supported by an arm. The arm may include one or more sections. In one example, a sensor (e.g., camera) may be supported at or near an end of an arm. The arm may include one or more sections, two or more section, three or more sections, four or more sections, or more sections. The sections may move relative to one another or a body of the medical console. The sections may pivot about one or more hinge. In some embodiments, the movements may be limited to a single plane, such as a horizontal plane. Alternatively, the movements need not be limited to a single plane. The sections may move horizontally and/or vertically. A sensor (e.g., camera) may have at least one, two, three, or more degrees of freedom. An arm may optionally include a handle that may allow a user to manually manipulate the arm to a desired position. The arm may remain in a position to which it has been manipulated. A user may or may not need to lock an arm to maintain its position. This may provide a steady support for a sensor (e.g., camera). The arm may be unlocked and/or re-manipulated to new positions as needed. In some embodiments, a remote user may be able to control the position of the arm and/or sensor(s) (e.g., cameras).

In some embodiments, one or more cameras may be provided at the second location. The one or more cameras may or may not be supported by the medical console. In some embodiments, one or more cameras may be supported by a ceiling 160, wall, furniture, or other items at the second location. For instance, one or more cameras may be mounted on a wall, ceiling, or other device. Such cameras may be directly mounted to a surface, or may be mounted on a boom or arm. For instance, an arm may extend down from a ceiling while supporting a camera. In another example, an arm may be attached to a patient's bed or surface while supporting a camera. In some instances, a camera may be worn by medical personnel. For instance, a camera may be worn on a headband, wrist-band, torso, or any other portion of the medical personnel. A camera may be part of a medical device or may be supported by a medical device (e.g., endoscope, etc.). The one or more cameras may be fixed sensors (e.g., cameras) or movable sensors (e.g., cameras). The one or more cameras may be capable of rotating about one or more, two or more, or three or more axes. The one or more sensor(s) (e.g., cameras) may include pan-tilt-zoom cameras. The sensors (e.g., cameras) may be manually moved by an individual at the location. The sensors (e.g., cameras) may be locked into position and/or unlocked to be moved. In some instances, the one or more sensor(s) (e.g., cameras) may be remotely controlled by one or more remote users. The sensor(s) (e.g., cameras) may zoom in and/or out. Any of the sensor(s) (e.g., cameras) may have any of the resolution values as provided herein. The sensor(s) (e.g., cameras) may optionally have a light source that may illuminate an area of interest. Alternatively, the sensor(s) (e.g., cameras) may rely on external light sources.

Images captured by the one or more cameras 145, 146 may be analyzed as described further elsewhere herein. The video may able analyzed in real-time. The videos may be sent to a remote communication device. This may allow a remote use to remotely view images captured by the field of view of the camera. For instance, the remote user may view the surgical site and/or any medical devices being used. The remote user may be able to view the medical personnel. The remote user may be able to view these in substantially real-time. For instance, this may be within 1 minutes or less, 30 seconds or less, 20 seconds or less, 15 seconds or less, 10 seconds or less, 5 seconds or less, 3 seconds or less, 2 seconds or less, or 1 second or less of an event actually occurring.

This may allow a remote user to lend aid or support without needing to be physically at the first location. The medical console and cameras may aid in providing the remote user with the necessary images and information to have a virtual presence at the first location.

Video analysis may occur locally at the first location 110. In some embodiments, the analysis may occur on-board a medical console 140. For instance, the analysis may occur with aid of one or more processors of a communication device 115 or other computer that may be located at the medical console. In some instances, the video analysis may occur remotely from the first location. In some instances, one or more servers 170 may be utilized to perform video analysis. The server may be able to access and/or receive information from multiple locations and may collect large datasets. The large datasets may be used in conjunction with machine learning in order to provide increasingly accurate video analysis. Any description herein of a server may also apply to any type of cloud computing infrastructure. The analysis may occur remotely and feedback may be communicated back to the console and/or location communication device in substantially real-time. Any description herein of real-time may include any action that may occur within a short span of time (e.g., within less than or equal to about 10 minutes, 5 minutes, 3 minutes, 2 minutes, 1 minute, 30 seconds, 20 seconds, 15 seconds, 10 seconds, 5 seconds, 3 seconds, 2 seconds, 1 second, 0.5 seconds, 0.1 seconds, 0.05 seconds, 0.01 seconds, or less).

In some embodiments, medical personnel may communicate with one or more remote individuals associated with a graphical code by a processor of the console. In some embodiments, the graphical code can be associated with a patient's identity.

A second location 120 may be any location where a remote individual 127 is located. The second location may be remote to the first location. For instance, if the first location is a hospital, the second location may be outside the hospital. In some instances, the first and second locations may be within the same building but in different rooms, floors, or wings. The second location may be at an office of the remote individual. A second location may be at a residence of a remote individual.

A remote individual may have a remote communication device 125 which may communicate with a local communication device 115 at the first location. Any form of communication channel 150 may be formed between the rep communication device and the location communication device. The communication channel may be a direct communication channel or indirect communication channel. The communication channel may employ wired communications, wireless communications, or both. The communications may occur over a network, such as a local area network (LAN), wide area network (WAN) such as the Internet, or any form of telecommunications network (e.g., cellular service network). Communications employed may include, but are not limited to 3G, 4G, LTE communications, and/or Bluetooth, infrared, radio, or other communications. Communications may optionally be aided by routers, satellites, towers, and/or wires. The communications may or may not utilize existing communication networks at the first location and/or second location.

Communications between rep communication devices and local communication devices may be encrypted. Optionally, only authorized and authenticated rep communication devices and local communication devices may be able to communicate over a communication system.

In some embodiments, a remote communication device and/or local communication device may communicate with one another through a communication system. The communication system may facilitate the connection between the remote communication device and the local communication device. The communication system may aid in presenting, on a remote communication device, a user interface to a remote individual about one or more possible medical procedures that may benefit from the remote individual's support.

A remote individual may be any user that may communicate remotely with individuals at the first location. The remote individual/user may lend support to individuals at the first location. For instance, the remote individual may support a medical procedure that is occurring at the first location. The remote user may provide support for one or more medical products, or provide advice to one or more medical personnel.

In some embodiments, the remote user may be a vendor representative. Medical products may be provided by one or more vendors. Typically, vendors may make arrangements with health care facilities to provide medical products. Vendors may be entities, such as companies, that manufacture and/or distribute medical products. The vendors may have representatives that may be able to provide support to personnel using the medical devices. The vendor representatives (who may also be known as product specialists or device reps), may be knowledgeable about one or more particular medical products. Vendor representatives may aid medical personnel (e.g., surgeons, surgical assistants, physicians, nurses) with any questions they may have about the medical products. Vendor representatives may aid in selection of sizing or different models of particular medical products. Vendor representatives may aid in function of medical products. Vendor representatives may help a medical personnel use product, or troubleshoot any issues that may arise. These questions may arise in real-time as the medical personnel are using a product. For instance, questions may arise about a medical product while a surgeon is in an operating room to perform a surgery. Traditionally, vendor representatives have been located at the first location with the medical personnel. However, this can be time consuming since the vendor representative will need to travel to the location of the medical procedure. Secondly, the vendor representative may be present but the vendor representative's help may not always be needed, or may be needed for a very limited time. Then, the vendor representative may have to travel to another location. It may be advantageous for a vendor representative to communicate remotely as needed with personnel at the first location. Thus, in systems and methods provided herein, the vendor representative may be a remote individual at a second location who may provide support remotely.

The remote users may be any other type of individual providing support, such as other medical personnel (e.g., specialists, general practice physicians, consultants, etc.), or technical support. Any description herein of vendor representatives may also apply to any other type of individual providing support, and vice versa.

In some embodiments, information about communications between remote users, such as vendor representatives, and the medical console (or any other device at the first location) may be collected and used for any of the processes described elsewhere herein. For instance, call data records may include one or more of the following: call start time, call end time, call duration, the identity of the individuals on the call (e.g., remote user identity, medical personnel identity such as identity of medical practitioner used to log into a medical console or the identities of all medical personnel present at a medical procedure), identity of the medical console making a call (e.g., each medical console may have a unique or semi-unique identity, which may or may not encode a health care facility identity and/or medical personnel identity), bandwidth on audio and video throughout the call, or any other factors. In some embodiments, factors, such as video or audio bandwidth may be indicative of the amount of activity that has occurred on the call. This may be indicative of the degree of active support provided by the vendor representative during the call.

A call quality may be provided for a call. For example, quantitative feedback, such as a numeral value for the call quality may be provided. For example, the remote user and/or on-site medical personnel may be provide feedback about the quality of the call itself. In one example, a score between 1 and 5 may be provided for the call. The users (e.g., remote users and/or on-site medical personnel) may be given an opportunity to provide the score after the call has been completed. This may be useful for assessing the functionality of the systems and methods provided herein.

In some instances, feedback may be provided for vendor representative (or other remote user) performance by the medical personnel. For example, a score of 1 to 5 may be provided for the vendor representative performance. In some instances, a vendor representative may be able to provide a score for the vendor representative's experience working with particular medical personnel.

In the systems and methods provided herein, a medical resource intelligence system may gather information collected at one or more locations (e.g., first locations). The medial resource intelligence may utilize video information, audio information, information from instruments that may be connected to a medical console, or information input by one or more medical personnel.

In some embodiments, a procedure preparation system may utilize information about an upcoming procedure, medical personnel involved, medical product preferences, or any other information in order to make recommendations for physical preparations that should take place at a location where the medical procedure will occur. The procedure preparation system may also identify remote users that may be needed to provide support.

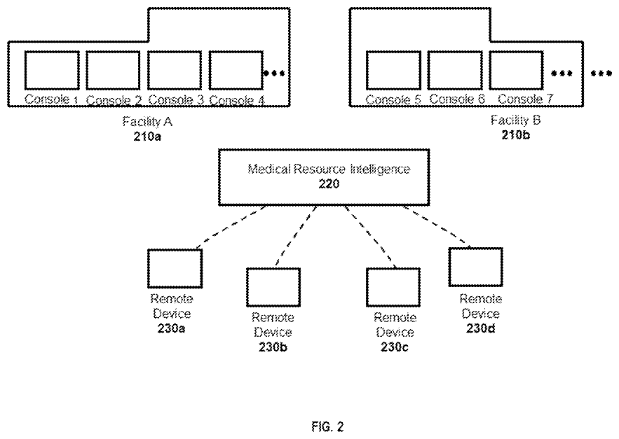

FIG. 2 shows an example of a procedure preparation system 220 configured to communicate with various devices, in accordance with embodiments of the invention.

In some embodiments, one or more health care facilities 210a, 210b may communicate with a medical resource intelligence system. The one or more health care facilities may be any type of health care facility such as those described elsewhere herein. The various types of health care facilities communicating with the medical resource intelligence system may be the same type of health care facility or different types of health care facilities. Such communications may occur simultaneously. Data from multiple health care facilities may be utilized by the medical resource intelligence system in providing outputs, such as remote user availability. In some instances, a procedure preparation system may communicate with a single health care facility, or only data from a single health care facility may be utilized in providing outputs, such as preferred remote user.

A health care facility may have one or more sources of information. Examples of sources of information may include one or more medical consoles. A medical console may be deployed at a location of a medical procedure. For example, one or more medical consoles may be deployed at an operating suite. In another example, one or more medical consoles may be deployed at a recovery room. One or more medical consoles may be deployed at an intensive care unit (ICU). One or more medical consoles may be deployed at a nurses' station. One or more medical consoles may be deployed in hallways. One or more medical consoles may be deployed at a clinician's office. In some instances, a plurality of consoles may be deployed anywhere throughout a health care facility. One or more of the consoles may be activated or in use throughout a health care facility. The one or more consoles may be actively communicating throughout a health care facility.

The one or more consoles may provide video and/or audio data that may be collected at the location. The one or more consoles may provide data from one or more auxiliary devices. The one or more medical consoles may provide data that may be input by one or more medical personnel. In some instances, data may be provided by one or more devices that are at the location of the medical procedure. For example, one or more additional video cameras or microphones may provide data in addition to the consoles. Any description herein of console communications may include information about one or more separate devices that may also provide data.

The one or more consoles may communicate with a medical resource intelligence system. The one or more consoles may communicate directly with a medical resource intelligence system. The one or more consoles may provide real-time updates to the medical resource intelligence system. In some embodiments, additional devices or information from the health care facilities may be provided to the medical resource intelligence system. For example, scheduling information, inventory information, usage information, terms of agreements with vendors, etc. may be provided to the medical resource intelligence system. Such information may be provided on a periodic basis (e.g., every week, every day, every several hours, every hour, every several minutes, every minute, every several seconds, every second, or every fraction of a second). Such information may optionally be provided to the medical resource intelligence system in real-time.

Optionally, the one or more medical consoles may not directly communicate with the medical resource intelligence system. The medical consoles may communicate with one or more system for the health care facility within which they operate. The health care facility systems may then provide relevant information to the medical resource intelligence system.

Any information provided to the medical resource intelligence system may be compliant with any regulations, such as regulations for privacy. Any communications to the medical resource intelligence system may be HIPAA compliant. In some embodiments, information may be automatically deleted or redacted as needed in order to comply with regulations or rules.

The medical intelligence resource system may also communicate with one or more remote devices 230a, 230b, 230c, 230d. The remote devices may be owned, operate, or used by one or more remote users. The one or more remote users may include vendor representatives, medical personnel, technical support, or any other type of remote user. The one or more remote devices may be any type of devices, such as tablets, smartphones, personal digital assistants, laptop computers, desktop computers, wearable devices, smart appliances, kiosks, or any other type of device.

In some embodiments, communications may occur between the consoles and the one or more remote devices. In some embodiments, the communications may occur directly between the consoles and the one or more remote devices. The communications may occur over a network, such as any type of network as described elsewhere herein. The existence, timing, duration, and identity of the devices (e.g., medical consoles or remote devices) may be logged and/or provided to the medical resource intelligence system. In some instances, the devices may include one or more geolocation device that may be used to determine the locations of the devices. The locations of the devices may be logged and/or provided to the medical resource intelligence systems as well.

The medical resource intelligence system may be implemented with aid of one or more devices. For instance, one or more servers may be utilized to implement the medical intelligence resource system. Optionally, one or more databases may be employed in order to store information. In some embodiments, cloud computing infrastructures may be utilized by the medical resource intelligence system. Optionally, peer-to-peer communications may be used in order to provide the functionality of the medical intelligence resource system. The medical resource intelligence, The systems and methods provided herein may utilize any type of infrastructure, such as P2P, Client/Server, Hybrid (P2P Multicast+Client Sever), P2P Multicast, Simulcast, Multicast, with help of HTTP ABR streaming protocols in addition to real time streaming protocols as a few different ways to stream data. The procedure preparation system may utilize one or more processors, individually or collectively, that may perform any of the steps described herein. The medical resource intelligence system may utilize non-transitory, tangible computer readable media which may comprise memory storage units that may comprise code, logic, or instructions to perform any of the steps herein

The medical resource intelligence system may utilize machine learning to provide any of the functionality described herein. One or more data sets may be provided. Machine learning data may be generated based on the data sets. The learning data may be useful for medical resource intelligence, such as identifying and forecasting use of medical resources, identities of individuals to contact, availability of the individuals to contact, evaluation of performance, performance metrics, video analysis, audio analysis, or other functions. The data from such functions may be fed back into the data sets to improve the machine learning algorithms.

One or more data sets may be provided. In some embodiments, data sets may advantageously include a large number of examples collected from multiple sources. In some embodiments, the medical resource intelligence system may be in communication with multiple health care facilities and may collect data over time regarding procedures and preparation for procedures. The data sets may include data about the patients, procedures performed, medical personnel involved, availability of medical personnel involved, products used, outcomes, communications information (e.g., identity of remote users, locations, timing, duration, type of support provided, etc.) and associated timing information. As medical personnel perform additional procedures and communicate with remote users, data relating to these procedures and communications may be constantly updated and added to the data sets. This may improve the machine learning algorithm and subsequent predictions over time. Operators of the system can provide preference lists of remote users such as vendors. The operators preference list can be associated with the specific operator. The vendor can provide a preference list of remote users (e.g., vendors) to be contacted if a preferred remote user is not available.

The one or more data sets may be used as training data sets for the machine learning algorithms. Learning data may be generated based on the data sets. In some embodiments, supervised learning algorithms may be used. Optionally, unsupervised learning techniques and/or semi-supervised learning techniques may be utilized in order to generate learning data.

In some embodiments, the machine learning may be used to improve object recognition. In some embodiments, video captured from one or more cameras during the medical procedure may be analyzed to detect medical products at the location. Optionally, audio data, medical records, or inputs by medical personnel may be used in addition or alternatively in order to determine medical products. In some embodiments, object recognition and/or sizing/scaling techniques may be used to determine the medical products at the location. A medical personnel may or may not provide feedback in real-time whether the medical product that was recognized using the video analysis was correct. In some embodiments, the feedback may be useful for improving medical product recognition in the future.

In some embodiments, the machine learning may be used to improve facial recognition or other ways of recognizing the identity and actions of the medical personnel. In some embodiments, video captured from one or more cameras during the medical procedure may be analyzed to detect identities of the medical personnel at the location. Similarly actions taken by the medical personnel may be recognized. Optionally, audio data, medical records, or inputs by medical personnel may be used in addition or alternatively in order to determine identity and/or actions of medical personnel. In some embodiments, object recognition and/or sizing/scaling techniques may be used to determine the medical personnel or their actions at the location. A medical personnel may or may not provide feedback in real-time whether the identities or actions that was recognized using the video analysis was correct. In some embodiments, the feedback may be useful for improving identity or action recognition in the future.

In some embodiments, the identity of the medical products that are actually used and timing of the use of the products during steps for a medical procedure may be predicted and/or recognized using a machine learning algorithm. In some embodiments, graphical code information, video information, audio data, medical records, and/or inputs by medical personnel may be used alone or in combination to determine the use of medical products and/or timing of use. Optionally, medical personnel may or may not provide feedback in real-time whether the identified use of the medical product and/or associated timing are correct. In some embodiments, the feedback may be useful for improving product use and timing predictions and/or recognition in the future.

As medical personnel are performing a medical procedure, the various steps for a medical procedure may be recognized using a machine learning algorithm. In some embodiments, graphical code information, video information, audio data, medical records, and/or inputs by medical personnel may be used alone or in combination to recognize the steps for the medical procedure that are being performed by the medical personnel. Machine learning may be useful for detecting and recognizing a series of steps for the procedure based on the collected information. Optionally, medical personnel may or may not provide feedback in real-time whether the detected steps are correct for the particular patient. In some embodiments, the feedback may be useful for improving step recognition in the future. In some embodiments, the feedback is associated with the graphical code that was identified by the console. In some embodiments, the graphical code is associated with the patient's identity.

Similarly, during a medical procedure, the timing for the various steps for a medical procedure may be recognized using a machine learning algorithm. In some embodiments, graphical code information, video information, audio data, medical records, and/or inputs by medical personnel may be used alone or in combination to recognize the timing of the steps for the medical procedure that are being performed by the medical personnel. For instance, the systems and methods provided herein may recognize the time at which various steps are started. The systems and methods provided herein may recognize a length of time it takes for the steps to be completed. The systems and methods provided herein may recognize when the next steps are taken. Machine learning may be useful for detecting and recognizing timing for a series of steps for the procedure based on the collected information. Optionally, medical personnel may or may not provide feedback in real-time whether the timing of the detected steps are correct for the particular patient. In some embodiments, the feedback may be useful for improving step timing recognition in the future. In some embodiments, the feedback is associated with the graphical code that was identified by the console. The console may communicate with the operator of the console using an audio and/or visual interface. Preferences of the operator can be altered via the audio and/or visual interface, such as the preferred remote user (e.g., vendor) list.

As described, machine learning may be useful for additional steps, such as recognizing individuals at the location (e.g., medical personnel) and items (e.g., medical products, medical devices) being used. The systems and methods provided may be able to analyze and identify individuals in the room based on the video frames and/or audio captured. For example, facial recognition, motion recognition, gait recognition, voice recognition may be used to recognize individuals in the room. The machine learning may also be utilized to recognize actions taken by the individuals (e.g., picking up an instrument, medical procedure steps, movement within the location, commands of the individual to the console, remote user preference of the individual). The machine learning may be utilized to recognize a location of the individual. The machine learning may also associate the actions of the individual and the location of the individual with the graphical codes of the medical devices used.

In some embodiments, the machine learning may utilize deep convolution neural networks/Faster R-CNN Nast NasNet (COCO). The machine learning may utilize any type of convolutional neural network (CNN) and/or recurrent neural network (RNN). Shift invariant or space invariant neural networks (SIANN) may also be utilized. Image classification, object detection and object localization may be utilized. Any machine learning technique known or later developed in the art may be used. For instance, different types of neural networks may be used, such as Artificial Neural Net(ANN), Convolution Neural Net (CNN), Recurrent Neural Net (RNN), and/or their variants.

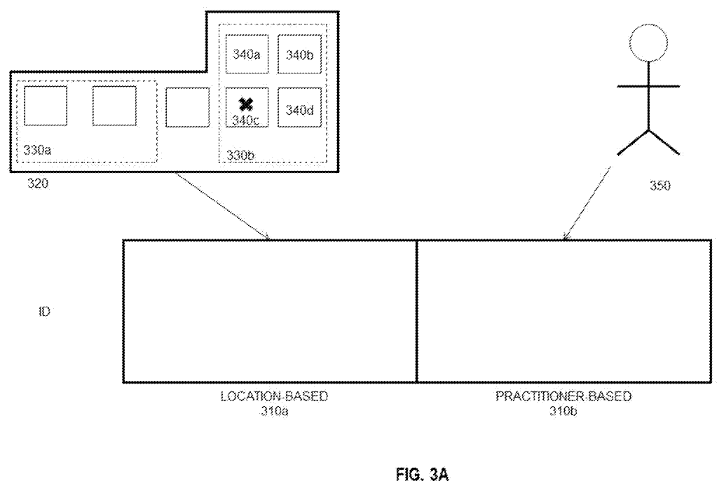

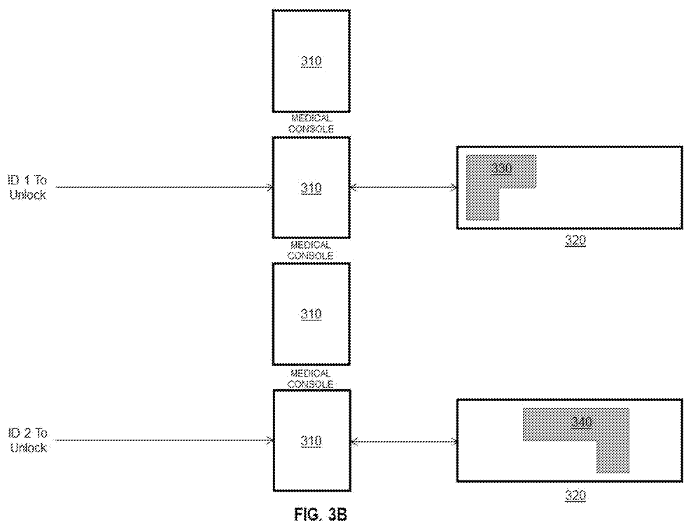

The machine learning utilized may optionally be a combination of CNN and RNN with temporal reference, as illustrated in FIG. 3. Input, such as cameras images, external inputs, and/or medical inputs may be provided to a tool presence detection module. The tool presence detection module may be configured to identify a graphical code of a tool. The tool presence detection module may communicate with EnodoNet. Training images may be provided for fine-tuning, which may provide data to EnodoNet. Additional input, such as camera images, external inputs, and medical images may be provided to EnodoNet. The output from EnodoNet may be provided to long short-term memory (LSTM). This may provide an output of a confidence score, phase/step recognition, and/or confusion matrix.

The machine learning may optionally utilize CNN for Multiview with sensors as illustrated in FIG. 3. In some embodiments, inputs, such as various camera views/medical images with sensors, and/or external imaging with sensors may be provided to a CNN learning module. This may provide output to feature maps, which may in turn undergo Fourier feature fusion. The data may then be conveyed to a fully connected layer, and then be provided to Softmax, and then be conveyed as an output.

In some embodiments, the machine learning as described and applied herein may be an artificial neural network (ANN) as illustrated in FIG. 3. The Multiview with sensors may be provided as illustrated. For instance, an input, such as one or more camera views/medical image or video with sensors may be provided to a predictive (computer vision/natural language processing) CV/NLP module. The output may be conveyed to an ANN module. The output from the ANN may be an analysis score or decision.

FIG. 3A shows an example of scene analysis utilizing machine learning, in accordance with embodiments of the invention. An input may comprise one or more camera views and/or medical image or video with sensors. The input may be provided to a module that may perform one or more functions, such as external input like vitals (e.g., ECG), tool detection, hand movement tracking, object detection and scene analysis, and/or audio transcription and analysis. The output from the module may be provided to a Markov logic network. Data from a knowledge base may also be provided to a Markov logic network. The output from the Markov logic network may be an output activity descriptor. In some embodiments, to unlock a medical console and/or access information using the medical console, a medical console identifier may be provided. The medical console identifier may be used to log into the medical console.

In some embodiments, medical consoles, such as those having any features or characteristics described elsewhere herein, may be located at a health care facility. The medical consoles may be used for different medical procedures by different individuals. The medical consoles may stay at a particular location in a health care facility (e.g., within a particular operating room), or may travel between different locations. For example, the medical console may be wheeled between different operating rooms, within the hallway, or any other location within the health care facility. The medical consoles may provide flexible access to communications and video data.

In some embodiments, the functionality of the medical console may not be accessed until an identifier is entered into the medical console. The medical console may essentially be a ‘blank slate’ that does not permit a user to access certain information or functionality until an identifier is entered into the medical console. A user may ‘log in’ to the medical console by using an identifier.

The identifier may have a location-based component 310a and/or a practitioner-based component 310b.

For example, the location-based component of the identifier may include information about where the medical console is being used. The location where the medical console is being used may include an identity of the health care facility 320 where the medical console is being used. For example, the identity of the health care facility may include a unique identifier for a particular hospital, clinic, assisted care facility, or any other type of health care facility. The location of the medical console may include a group or department 330a, 330b within the health care facility. In some embodiments, the location of the medical console may include a particular physical room or region within the health care facility 340a, 340b, 340c, 340d (e.g., operating room, examination room, recovery room, nurses' station, etc.). Any of these levels of location-based identifiers are provided by way of example only, and may not be required. Any combinations of these identities may be incorporated into the location-based component of the identifier.

For example, the identity of the various locations may be provided as an alphanumeric identifier that uniquely identifies the location. For instance, the identifier for Hospital ABCD may be HospitalABCD, or may be a random or pseudorandom string that identifies Hospital ABCD (e.g., H1dj29Gnml). In some instances, the various level of location may each have their own unique identifier. The identifiers for various levels may be appended to one another. For example location-based ID=hospitalID+operatingroomID. For instance, a location-based ID may look something like HospitalABCD-OperatingRoom26. In another example, a location-based ID may look something like HospitalABCD-Cardiology-OperatingrRoom26. In other embodiments, the various components of the identifier may undergo any type of calculation or manipulation (e.g, hash, algorithm, etc.) to yield a location-based identifier that is unique to that location. For example, the resulting ID may look something like aagh490go51xg, but may be unique to a particular location. Using the identification, a remote user associated with a barcode on medical equipment can obtain relevant information about the location, medical personnel, or patient without needing to initiate a search. The identification can also be used by the console to identify remote users based on location preference or individual preference.

In some embodiments, the medical consoles may be dedicated to certain locations, such as certain health care facilities. In some instances, the identifier for that fixed location may be pre-encoded into the medical console. For example, if the medical console is always used at Hospital ABCD, the portion of the identifier for that health care facility may already be encoded or programmed into the login identifier. For instance, when a user is given an option to enter an identifier, the prompt may appear like: HospitalABCD-______.

A practitioner-based component of the identifier may include information about an identity of an individual using the medical console. The identity of an individual using the medical console may refer to an identity of the medical personnel who is performing the procedure that the medical console is supporting. For example, the practitioner-based component may refer to an identity of a surgeon performing a procedure. In some instances, the identity component may refer to an individual who is in charge of the procedure, even if the individual is not the one physically logging into the console or entering data into the console. For example, the identity of the component may refer to a surgeon's IDS, even if one of the nurses is logging into the medical console prior to the procedure. In other instances, the identity may include any other individual present at the medical personnel, such as a nurse, surgical assistant, or any other individual. Any of these identifiers are provided by way of example only, and may not be required. Any combinations of these identities may be incorporated into the practitioner-based component of the identifier.

For example, the identity of the medical practitioner may be provided as an alphanumeric identifier that uniquely identifies the location. For instance, the identifier for surgeon William Jones may be William Jones, or may be a random or pseudorandom string that identifies William Jones (e.g., A1dj29GnMD). In some instances, each practitioner may each have their own unique identifier.

The identifiers for various components may be appended to one another. For example, the location-based identifier and the practitioner-based identifier may be appended to one another: e.g., loginID=locationID+practitionerID. For instance, a login at a console may utilize an identifier that may look something like HospitalABCD-OperatingRoom26-WilliamJones. In other embodiments, the various components of the identifier may undergo any type of calculation or manipulation (e.g, hash, algorithm, etc.) to yield an identifier that is unique to that location/practitioner combination.

FIG. 3B shows an example of how medical console identifiers may affect functionality of medical consoles, in accordance with embodiments of the invention.

In some embodiments, a medical console may be provided at a health care facility 310. Prior to an individual logging into the medical console, the medical console may essentially be a ‘blank slate.’ The medical console may not be able to access certain information or resources prior to an individual logging in. In some instances, an individual may not be able to use the medical console until the individual logs in. The individual may not be able to able to perform some or any of the features as described elsewhere herein.

In one example, a login identifier (e.g., ID1) may be entered into the medical console, as illustrated. When the login identifier is entered, the medical console may be able to access one or more resources 330. In some embodiments, the resources may be a subset of all resources that may be possible 320. In some instances, the console may only be able to access a portion of the subset of all resources based on the login identifier. For instances, based on the health care facility, location within the health care facility, and/or medical practitioner identifier, only relevant information may be accessible by the medical console. The console can be configured to associate the login identifier of a medical practitioner to a list of preferred remote users. Once a graphical code of medical equipment has been recognized by the console via a camera or scanner, the console can be configured to automatically provide options to the medical practitioner to initiate a call to a preferred remote user associated with the medical equipment.

In one example, a central repository 320 of information may be provided for a health care facility, laboratory, group/department, or room. The central repository may have a cloud-based infrastructure, a P2P infrastructure, or any other infrastructure or characteristics as described elsewhere herein. The central repository may operate on one or more servers. The central repository may comprise one or more processors that may individually or collectively perform any of the steps described herein. The central repository may comprise non-transitory, tangible, computer readable media, which may comprise memory units, which may store code, logic, or instructions for performing one or more of the steps described herein.

The central repository may comprise any type of information relating to the health care facility, group/department, operating room, medical personnel, patient information, previous remote call/communication information, historical information, business information, or any other type of data. For instance, the central repository may comprise information about medical personnel preferences, medical procedures, steps for medical procedures, associated timing information, clinical outcome information, or any historical information.

When a login identifier (e.g., ID1) is used to unlock the medical console, the medical console may be provided with access to one or more resource 330 such as one or more online resource. The online resource may be information or data that may be stored at or accessible by the central repository. In some instances, the accessible resources may be a subset of all information provided at a central repository. The accessible resources may be resources that a medical personnel whose identifier is provided at the login is authorized to access. This may be useful for providing patient privacy or limiting access to irrelevant or private information. In some instances the medical console may provide access to information that is only relevant to the medical personnel whose identifier is provided at login.

In some instances, the medical console may provide access to information that is only relevant for the location identifier that is presented at login. For example, a central repository may optionally comprise information across multiple health care facilities. The medical console may only be able to access information from the health care facility corresponding to the location identifier entered at the login.

When one or more users are done using a medical console, an individual may log out of the medical console. When logout has occurred, the medical console return to being a ‘blank slate.’ The medical console may not be able to access certain information or resources anymore. In some instances, another individual may not be able to use the medical console until the individual logs in. The individual may not be able to able to perform some or any of the features as described elsewhere herein.

At a later time, another login identifier (e.g., ID2) may be entered into the medical console, as illustrated. When the second login identifier is entered, the medical console may be able to access one or more resources 340. In some embodiments, the resources may be a subset of all resources that may be possible 320. In some instances, the console may only be able to access a portion of the subset of all resources based on the login identifier. For instances, based on the health care facility, location within the health care facility, and/or medical practitioner identifier, only relevant information may be accessible by the medical console. If the two login identifiers are different (e.g., if ID1 is different from ID2), then different subsets of information may be accessed (e.g., 330 and 340 may be different from one another). The information accessed by different login identifiers may or may not have some overlap. The information accessed by different login identifiers may or may not overlap in their entirety, or may include different information.

When another login identifier (e.g., ID2) is used to unlock the medical console, the medical console may be provided with access to one or more resource 340 such as one or more online resource. The online resource may be information or data that may be stored at or accessible by the central repository. In some instances, the accessible resources may be a subset of all information provided at a central repository. The accessible resources may be resources that a medical personnel whose identifier is provided at the login is authorized to access. This may be useful for providing patient privacy or limiting access to irrelevant or private information. In some instances the medical console may provide access to information that is only relevant to the medical personnel whose identifier is provided at login.

In one example, medical personnel procedure practices (e.g., surgeon prep cards) may be accessed by the medical console. In one example, a first practitioner may login with his identifier (e.g., ID1=locationid+practitionerid1). The first practitioner may subsequently log out and a second practitioner may log in with his identifier (e.g., ID2=locationid+practitionerid2). In such a situation, when logged in, the first practitioner may be able to access his own personal preferences or records without being able to access the second practitioner's personal preferences or private records. Similarly, when logged in, the second practitioner may be able to access his own personal preferences or records without being able to access the first practitioner's preferences or private records. In some instances, there may be some overlap between information that the first and second practitioners are able to log in.

In another example, different logins may occur at different locations. The different locations may comprise different rooms within the same health care facility, or across different health care facilities. In some instances, the information accessible may or may not be different based on the different locations. For example, for different operating rooms, the resources accessible by a medical console may or may not be the same. For different health care facilities, the resources accessible by a medical console may include at least some information that does not overlap.

Medical personnel may have different data or preferences at different locations. For example, a medical practitioner may have a first set of preferences at a first hospital and a second set of preferences at a second hospital. This may be due to practitioner preference at different health care facilities, or different health care facilities may have different rules or resources (e.g., products) available. For example, a medical practitioner may have a first surgeon prep card when logging in at a first location, and a second surgeon prep card when logging in at a second location.

For example, medical personnel procedure practices (e.g., surgeon prep cards) may be accessed by a medical console. In one example, a practitioner may login with his identifier (e.g., ID1=locationid1+practitionerid). The practitioner may subsequently log out. The practitioner may access the same medical console or a different medical console at a different location (e.g., ID2=locationid2+practitionerid). In such a situation, when logged in at the first location, the practitioner may be able to access a first set of personal preferences or records associated with the first location without being able to access the practitioner's personal preferences or private records associated with the second location. Similarly, when logged in at the second location, the practitioner may be able to access his own personal preferences or records associated with the second location without being able to access the practitioner's preferences or private records associated with the first location. In some instances, there may be some overlap between information that the practitioner can access at the first and second locations.

In some instances, each location ID may pull up different location preferences. For example, each health care facility may have its own preferred vendors, products, vendor representatives, procedure type protocols, instruments, disposables, rules, etc. In some instances, the location ID may instill a set of rules or preferences that may or may not trump practitioner preferences if there is a conflict. In some instances, practitioner preferences may be automatically updated to conform to location rules.

As previously described, one or more cameras or scanners 510 may be provided at a location of a medical procedure. The one or more cameras or scanners may include cameras on a medical console, supported on a ceiling, a boom, an arm, a wall, furniture, worn by medical personnel, or any other location. Multiple cameras or scanners may optionally be provided. The video collected by the cameras may be aggregated and/or analyzed by a video analysis system. The graphical codes identified by the scanners may be analyzed and correlated with the data (e.g., products identified) from the video collected.

The one or more cameras or scanners may individually or collectively capture images of the graphical codes of medical products 530a, 530b, 530c, 530d that may be used at the location. In one example, one or more cameras may individually or collectively capture an image of graphical codes of medical products that may be provided at a single location, such as a tray 520.

The video analysis system may be able to recognize the medical products that are provided. The medical product may be recognized in accordance with medical product type (e.g., stent), or may be recognized specifically to the model level (e.g., Stent Model ABCD manufactured by Company A). In some embodiments, the medical products may have graphical codes, such as QR codes, barcodes (e.g., 1D, 2D, 3D barcodes), symbols, letters, numbers, characters, shapes, sequences of lights or images, icons, or any other graphical code that may be useful for identifying the medical product. The cameras may capture images of the graphical codes, which may be useful for identifying the product type, specific product model, and/or specific product (e.g., tracked to the individual product, or batch/group).

In some embodiments, audio information may be collected as well. For example, speech by medical personnel may be analyzed to detect words that may refer to medical products. In some instances, the sound of medical products being used may be analyzed and recognized.

Medical records, surgeon prep cards, inputs by medical personnel, or any other sources may be used in recognizing the medical products that are provided at a procedure.

Additionally, the systems and methods provided herein (video, audio, records, prep cards, inputs, etc.) may be used to track usage of the medical products. For instance, the video may capture a medical personnel lifting a medical product and using it at a step during the procedure. The systems and methods provided herein may be able to recognize different steps of the procedure. The steps of the procedures may be predicted or known. In some instances, the steps of the procedure may provide context in trying to determine whether a particular medical product is being used. For example, if it is determined that a particular step is occurring, and that the step would require the use of a particular instrument, then the product that is imaged as being used may be interpreted within that context.

The timing and details regarding the actual use of the medical product may be recognized. Support given by a vendor representative at that time may also be recognized. In some embodiments, the timing and steps taken during the procedure may be used to determine efficacy of the product and/or support.

In some embodiments, the information may be collected passively without requiring any specialized input by medical personnel. For example, the images of the products may be automatically calculated and recognized.

Alternatively, or in addition, medical personnel may provide some input or perform an action that may aid in detecting the products provided and/or used. In some instances, medical personnel may speak about the products that they are using. For example, as a medical personnel performs a step, the medical personnel may include information about the step and/or the product that is being used. One or more microphones may connect information and be able to translate the speech into text and/or recognize the products described.

In another example, medical personnel may scan the medical products to be used. For example, they may use a scanner to scan one or more graphical code provided on the product. This may occur prior to the medical procedure or at the beginning of the medical procedure. In some instances, scanning may occur as products are used as well to track the use of the products.

Optionally, the devices or wrappers for the devices may include RFID or other type of near field communication. One or more scanners or readers may be provided to detect the communications coming from the device to recognize product usage.

Any of the systems and methods provided herein may be used for product recognition. The product recognition may be provided substantially in real-time as the products are being used at a location. For example, during a medical procedure, the products may be recognized prior to usage, and the usage of the products may be tracked.

The product recognition may be used to update a preference card. For example, if a product that is listed on the preference card is used, it may be used to affirm that the practitioner still uses the product listed on the preference card. If the product listed on the preference card is not used, it may be an indicator that the product is no longer needed at the outset of a procedure and that the product can be removed from the practitioner's preference card. In some instances, a single non-use of a product may be sufficient to update the preference card. Alternatively, a single non-use of the product may not be sufficient to update the preference card. An established pattern of non-use over time may be required in some instances to update the preference card. The use or non-use of a product may be analyzed from the historical records to determine whether to update the preference card.

In another example, if a product that is not listed on the preference card is used, the system may recognize the usage of a product that is not initially listed on the preference card. It may be an indicator that the product is typically needed for the procedure and the product can be added to the practitioner's preference card. In some instances, a single use of a product may be sufficient to update the preference card. Alternatively, a single use of the product may not be sufficient to update the preference card. An established pattern of use over time may be required in some instances to update the preference card. The use or non-use of a product may be analyzed from the historical records to determine whether to update the preference card.

In some embodiments, the function of the products and/or equivalents may be analyzed or recognized. For example, if a product is listed but not used, but a functionally equivalent product is used instead, this may provide a higher likelihood that the initial product is replaced with the functionally equivalent product that is used. When determining whether to update a preference card, whether a functionally equivalent product is used instead may be analyzed.

In some instances, data from product recognition may be used to affect the preference cards. In some instances, the data from the preference cards may or may not be used to affect product recognition. For example, when a video capture system captures images of products, they may use information from the preference cards to provide context in aiding in object recognition. For example, based on a practitioner's preferences, it may be known that the practitioner prefers to use Products 1, 2, and 3 for the type of procedure that is scheduled. The video capture system may capture images of various products that have been prepared for a procedure. If there is uncertainty about the identity of the products, the products listed in the practitioner's preference card may aid in recognizing the object. For example, if it is uncertain whether a product on a tray is Product 1 or Product 4, by knowing that the practitioner's preference card currently lists Product 1, product may be recognized as Product 1. In some instances, bi-directional communication may occur between product recognition and preference card listings.

In some embodiments, use of artificial learning and/or machine learning may be used to determine the best products (e.g., for a particular procedure and give recommendations. One or more analytical models utilizing any machine learning techniques described herein or known in the art may be used to recommend one or more medical product for a particular procedure type. In some instances, recommendations may be provided based on available products for a particular health care facility or group/department. The systems and methods provided herein may give details about success rates when a particular product is used along with diagnosis and prognosis details.

The success rates and/or recommendations may include details about what a particular brand (i.e., vendor) is provided for the product, make or model for the product, or any other factors or details relating to the product.

Furthermore, information such as time taken during surgery for different products (e.g., different types of products, different brands of products, different make/models of products, etc.) Time taken during surgery may relate to a totality of time to complete the procedure. Time taken during surgery may relate to the timing of individual steps taken during the procedure. In some instances, the timing information during the surgery may be ted to success and/or failure rates.

In some instances, any number of factors and parameters may be compared and analyzed. The various combinations of factors and parameters may be considered when providing a recommendation for a product to be used. The various combinations of factors and parameters may be considered when providing success and/or failure rates for various products given a set of circumstances. For instance, a relational matrix may be utilized with any number of combinations of tools used (e.g., tools type, brands, make/models), demographic information about a patient (e.g., age, sex, ethnicity, etc.), success/failure rate, timing information (e.g., turnaround time, timing of steps), health care facility, and/or accuracy of procedure or step.

The analytical model (which may utilize artificial or machine learning), can be present and/or operate locally on a medical console. Optionally, the analytical model may be present and/or operate on the cloud, shared across multiple consoles or with one or more remote participants. Any architecture described herein or known in the art may be used.

A procedure preparation system may receive or access information that may be useful in allowing various resources to be prepared for a medical procedure. In some instances, it may be desirable for various medical resources to be prepared before a medical procedure is scheduled to start. For example, before a surgery, the operating room (location) may be determined, the medical may be laid out or provided at the location, the proper medical personnel (e.g., surgeons, surgical assistants, nurses) on-site may be determined and present. Similarly, one or more remote users, such as vendor representatives, specialists or other types of remote users may be identified, contacted or scheduled ahead of time. Alternatively, remote users may be identified and/or contacted during the procedure.

In some embodiments, various inputs may be provided to the procedure preparation system. Such information may automatically be provided based on historical or data stored at a central repository. In some instances, the information may be based on information that is updated in real-time.

For example, scheduling information at a health care facility may be updated in real-time. As procedures are occurring in various operating rooms, the predicted time of completion of the procedures may be assessed in real-time. Further description of scheduling is provided in greater detail below. Based on the real-time scheduling information, it may be determined which locations are available for the medical procedure. For example, if a medical procedure was originally planned for a particular location, but the prior procedure in that location is running long, the system may be able to assess and find a location that will be available at the required time and update/select the location for the present medical procedure. Thus, the procedure preparation system may provide an up-to-date location for the procedure.

In some instances, practitioner preference cards may be accessed, or any other indicators of practitioner preferences. For example, various medical products that the practitioner prefers may be stored in a preference card. The preferred products for particular procedure types may be stored. In some instances, the medical practitioners may have other preferences, such as location preference, team member preferences, remote user preferences, or any other preferences. Such preferences may optionally be automatically updated as described elsewhere herein. Alternatively, or in addition, the preferences may be updated manually. One or more members of the team may prepare the medical resources prior to the procedure. For example, before a medical procedure takes place, the tools for the procedure may be laid out and prepared. The appropriate medical personnel who will be participating may be prepared and scrubbed in for the procedure. In some the procedure preparation system may provide a list of tools and/or personnel for the procedure. This may allow the tools and/or personnel to be prepped. In some instances, a team may gather and/or prep the products that are required before a medical practitioner performing the procedure arrives.

The medical personnel may be informed about the procedure and/or the specific steps that are planned for the procedure.

Optionally, medical personnel may be able to view information about the procedure and steps before the procedure begins. For example, prior to performing the procedure, a medical console may provide information about preferred remote users associated with a graphical code located on medical equipment, suggested steps, and/or estimated timing for various steps of the procedure for the particular patient. In some instances, recommended details about techniques or steps may be provided. In some cases, recommended remote users may be provided based on the availability of the preferred remote users. A medical practitioner may view a list of preferred vendors or have a list of preferred remote users relayed to them via an audio interface. A medical practitioner may view a list of steps and be permitted to select a step to view further details and/or example videos. This may allow a medical practitioner to prepare for a procedure immediately beforehand. The medical practitioner may provide a graphical code to the console via camera or scanner and immediately be provided a list of preferred remote users associated with the graphical code. Alternatively, the console may automatically identify a graphical code on medical equipment in the room. This may provide a last-minute refresher for the medical practitioner before performing a procedure. In some instances, the medical practitioner may be able to focus on particular details as needed.

Optionally, medical personnel may be able to input a graphical code from medical equipment to be used into the console via a camera or scanner. The graphical code can be cross referenced against the identity of the medical personnel to provide a list of preferred remote users based on the availability of the remote user and preferences input by the medical personnel. The console can be configured to comprise voice recognition to identify the medical personnel. The console can be configured to provide options to the user through a screen as well as through an audio menu. The console can be configured to obtain instructions from the medical personnel via a microphone. The console can be configured to automatically connect the medical personnel to a preferred remote user upon receipt of a graphical code from medical equipment.

Optionally, the information may be accessible during the medical procedure through a display and/or an audio interface. A video capture system may be used to capture images of the steps during a medical procedure. The progress and/or timing of the steps completed may be tracked. Accordingly predicted timing of subsequent steps may be updated in real-time. Furthermore, if certain conditions are detected during the procedure, the recommended steps themselves may be adjusted. For example, if a surgeon inadvertently cuts an artery, the systems and methods provided herein may recognize this, and the recommended steps may be adjusted to reflect the change in condition. The medical personnel may be able to view the recommended steps during the procedure. Such information may provide useful guidance during a medical procedure. The medical personnel may be alerted to the recommended steps via an audio alert. The operator may be able to interact with the console using a microphone. The console can be configured to call a remote user based on the conditions detected during the procedure and/or an oral command from the medical personnel obtained via microphone.

The information about the procedure, recommended steps, and/or timing may be displayed on a display device. The display device may be at or on the medical console. The display device may be integrated into the medical console. The console may be configured to interact with a operator via an audio system. A microphone may be used to obtain inputs such as selections and/or answers from the operator.

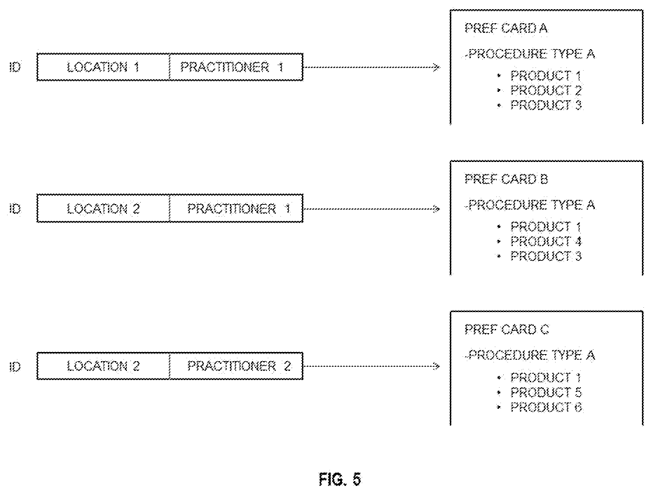

FIG. 5 shows an example of how practitioner preference cards may be accessed, in accordance with embodiments of the invention. The practitioner preferences cards (e.g., surgeon preference cards, surgeon prep cards, etc.) may show the practitioner's preferences for resources (e.g., products, remote users, medical personnel on location, operating rooms, etc.) for various medical procedures. For example, the practitioner's preferences for a first procedure may be different from the practitioner's preferences for a second procedure.

The practitioner preferences may optionally be different at different locations. For example, a practitioner preference card at a first location may list different medical resources from a practitioner preference card at a second location, even if they are the same practitioner.

For example, as illustrated, when a user logs in with an identifier for Location1+Practitioner1, the preference card may list Products 1, 2, and 3 for Procedure Type A. When the same user logs in at a different location, with an identifier for Location2+Pracitioner1, the preference card may list different remote users for the same procedure, such as Remote User 1, 2, 3 for Product 1. This may be due to practitioner personal preferences at different locations, or different rules imposed at the different locations. This may be due to the availability of the remote user. The availability of the remote user may be provided by the remote user or may be automatically identified based on procedures occurring in the same medical facility or procedures occurring in other medical facilities in which the remote user in already scheduled or involved in.

In one example, the rules for a particular location may change. For example, a health care facility may initially prefer remote user 1, but then later update the list to include remote user 2. When changes in rules are imposed, then the practitioner preference cards may be automatically updated. This may occur when the rules at the health care facility trump personal preferences. In some instances, the proposed change may be displayed to the practitioner to approve or reject. In some instances, when the changes at the health care facility level are merely suggestions, an option to change may be presented to the practitioner to approve or reject.

Even when a location is the same, different medical practitioners may have their own preferences. For example, as illustrated, when a user logs in with an identifier for Location2+Practitioner1, the preference card may list Products 1, 3, and 4 for Procedure Type A and preferred remote users for each of Products 1, 3, and 4. When a different user logs in at the same location, with an identifier for Location2+Pracitioner2, the preference card may list different products for the same procedure, such as Products 1, 5, and 6 for Procedure Type A and preferred remote users for each of Products 1, 5, and 6.

In some embodiments, individual practitioners may specify their preferences. In some examples, practitioners may enter their preferences into their preference cards. In another example, a video capture system, such as those described elsewhere herein, may be used to initially establish the practitioner's preferences and initially populate a preference card, for example by obtaining graphical codes of the products used.

However, practitioner preference may change over time. The practitioners may be introduced to new products that they may end up preferring, or the locations where they were may impose new rules that may affect the availability or desirability of particular products. In some instances, the practitioners may experience different clinical outcomes with different products. However, it may be burdensome to try and update the practitioner's preferences in real-time, or in a timely manner. Thus, it may be desirable to have systems and methods that may automatically update a practitioner's preferences, as changes in preference are detected. It may be desirable to permit the system to detect changes in preference depending on usage of various products in real-time. It may be advantageous to permit the practitioner preferences to be updated without requiring any user input.

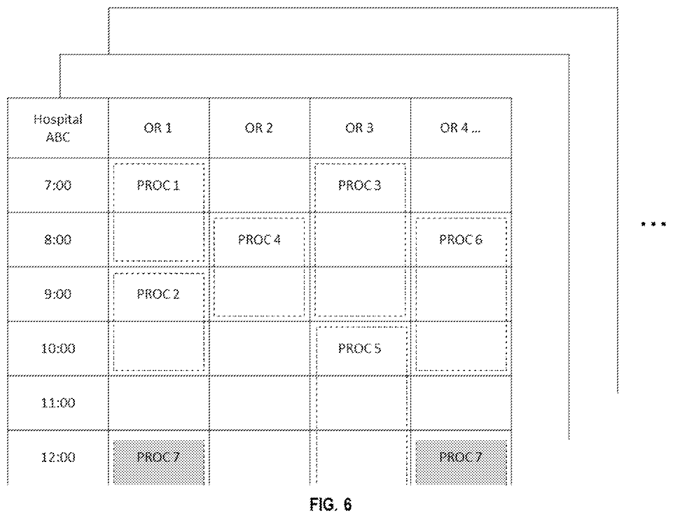

FIG. 6 shows a illustration of how scheduling may be updated in real-time, in accordance with embodiments of the invention. As previously described, providing up-to-date scheduling may be useful for forecasting the availability of preferred remote users.

In some cases, the captured videos and/or the one or more timing parameters associated with the plurality of videos may be used to update scheduling information for a particular remote user in real time. For example, as shown in FIG. 6, in Hospital ABC, there may be multiple locations where one or more surgical operations may be schedule. The multiple locations may include multiple operating rooms (e.g., OR1, OR2, OR3, OR 4, etc.). Depending on the preferences of the operator, one remote user may be listed as the preferred remote user for multiple locations. The system can coordinate the availability of the remote user between multiple locations.

The scheduling information may include timing information, such as time of day for a particular day. In some instances, the scheduling information may be updated in real time. Updating scheduling information in real time may enable medical operators, practitioners, personnel, or support staff to anticipate changes in a timing associated with a performance or completion of one or more steps of a surgical procedure and to prepare for such changes accordingly. Such real time updates may provide medical operators, practitioners, personnel, or support staff with sufficient time to prepare operating rooms or medical tools and medical instruments for one or more surgical procedures. Such real time updates may also allow medical operators, practitioners, personnel, or support staff to coordinate the scheduling of a plurality of different surgical procedure within a health care facility and to manage the resources or staffing of the health care facility based on the latest timing information available. Scheduling information may be available for the current day, upcoming day, the next few days, the next week, the next month, etc. The scheduling information may be updated in real-time, or may be updated periodically (e.g., daily, every several hours, every hour, every 30 minutes, every 15 minutes, every 10 minutes, every 5 minutes, every minute, every second, or more frequently). The scheduling information may be updated in response to an event. The scheduling information may include information about a procedure that may occur at the various locations. The scheduling information may include information about when and where each scheduled surgical procedure at a health care facility will be performed for any given date.

In some cases, the scheduling information may include additional information about procedures. For example, Procedure 1 may be scheduled to occur at 7:00 AM in OR1. Procedure 4 may be scheduled to occur at 8:00 AM in OR 2. Procedure 2 may be scheduled to occur at 9:00 AM in OR 1. The procedures may be of different type. The estimated length of time for each procedure may or may not be provided. The estimated length of time for each procedure may be updated based on timing information derived from the plurality of videos captured by the plurality of imaging devices.

In some cases, if an actual timing for one or more steps of Procedure 2 is delayed or takes longer than a predicted timing for the one or more steps of Procedure 2 (i.e., if Procedure 2 takes longer than predicted or estimated), then an estimated completion time for Procedure 2 may be updated. Based on the updated estimated completion time for Procedure 2, Procedure 7 may be rescheduled for a different time or a different operating room at the same time. For example, Procedure 7 may be moved from OR 1 to OR 4. The timing for Procedure 6 may be simultaneously tracked, and Procedure 7 may be moved to OR 4 if the timing for Procedure 6 is not running over.

The system may be able to determine that Procedure 2 is running long so that the location for Procedure 7 needs to change. This may advantageously allow Procedure 7 to be prepared and set up a location. In some instances, time to prepare may be required, and it may be beneficial to know in advance if the location needs to change. The systems and methods provided herein may provide real-time updates so that it may be apparent sooner if a procedure will likely run over and if a location needs to be changed.

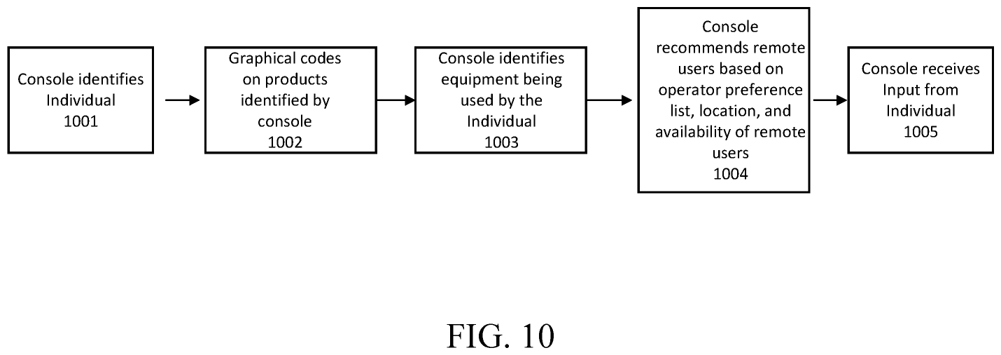

FIG. 10 shows a flow chart of an example of how an individual may use a console as described herein. An individual, such as a physician, may enter a room where a procedure may be set to take place. The console can recognize the individual, 1001, using, for example, facial recognition, voice recognition, input of a preference card, proximity of the console to a RFID carried by the individual and/or a sign-in by the individual. The console can recognize an individual automatically as they enter the room. The console can recognize products in the room, 1003, for example by object recognition, graphical code recognition, and/or RFID. A graphical code and/or RFID can be used by the console to identify a patient. The individual can scan graphical codes on the products. The console can recognize products automatically when placed in the room. The console can be configured to associate the recognized products with lists of preferred remote users for the procedure 1004. The preferred remote users can be based on lists of remote users provided by the operator and/or preferences provided by the hospital. The remote users can be vendors, physician specialists, consultants, etc. The console can communicate the preferred remote user to the operator and receive input from the operator, 1005, to contact the preferred remote user. The console can communicate with the operator without requiring a physical input (e.g., touchless). The operator can sign into the console without requiring a physical input (e.g., touchless).

The console can comprise one or more cameras and/or scanners. The console can comprise one or more microphones. The console can comprise one or more speakers. The console can comprise a display. The console may be able to analyze and identify individuals in the room based on the video frames and/or audio captured. For example, facial recognition, motion recognition, gait recognition, voice recognition may be used to recognize multiple individuals in the room in addition to the operator. In some cases, the systems and methods of the present disclosure may be used to enhance audio quality using natural language processing (NLP). The voice recognition can comprise natural language processing. Natural language processing (NLP) can comprise a self-learning environment to facilitate analysis and categorization of an individual's voice using statistical language understanding techniques, similarity-matching algorithms, machine learning algorithms, and the like. NLP may comprise manipulating and/or processing natural language such as speech and text in order to derive information or data associated with the speech and/or text (e.g., information about upcoming critical steps in a surgical procedure, a certain type of tool needed to complete a surgical step, or a specific type of support needed for a particular surgical step).

The console may comprise a medical resource intelligence system. The medical resource intelligence system can comprise machine learning. The machine learning may be utilized to recognize actions taken by the individuals (e.g., picking up an instrument, medical procedure steps, movement within the location, commands of the individual to the console, remote user preference of the individual). The machine learning may be utilized to recognize a location of the individual. The machine learning may also associate the actions of the individual and the location of the individual with the graphical codes of products used. The console can be configured to follow the movements of an individual and recognize which product the individual is using. The console can prompt the individual with an option to call a preferred remote user when the console identifies that an individual has been using a particular product for longer than a pre-determined range of time. The prompt can be an audio prompt. The medical resource intelligence system can apply a machine learning algorithm to determine an expected amount of time that a product is used. The machine learning algorithm can adjust the time based on observation of individuals. The console can be configured to respond to voice commands of the individual. The console can be configured to automatically contact a preferred remote user when a product is being used by an individual. The console can use motion recognition and object recognition to monitor the length of time an individual is using a product. Motion recognition can be used to identify when the individual may be struggling with a product or a part of a procedure. The console can be configured to automatically contact a preferred remote user when the console detects that an individual is struggling or there is an error in the procedure. The console can be configured to automatically contact a preferred remote user when an individual exceeds an expected amount of time using a product. The console can be configured to automatically contact a preferred remote user when a certain part of a procedure is initiated. The console can be configured to prompt the individual for confirmation that the remote user should be contacted.

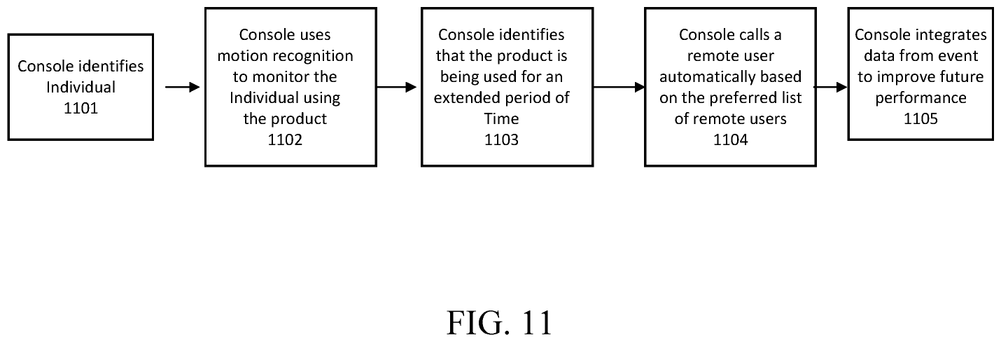

FIG. 11 shows a flow chart of an example of how the console can be used to monitor a procedure. The console can be used to identify the individuals in a room and the products in a room 1101, as described herein. The console can use motion recognition to monitor an individual using a product in a procedure 1102. The console can record the time that the individual is using the product and identify if the individual is using the product for an extended period of time 1103, indicating that the individual may be struggling with the product. The console can automatically contact a preferred remote user 1104. The contact can be via a video-conference which relays to the remote user a video of the procedure in real time. Alternatively, the console can prompt the individual through a touchless, audio interface (e.g., voice recognition) to confirm that a preferred remote user should be called 1104. The console can integrate data from the event to improve future performance 1105. The individual can provide feedback to the console through the touchless audio interface (e.g., voice recognition) to confirm a remote user, select a new remote user, cancel the prompt to call the remote user, etc.

In some embodiments, the remote user support may be needed at a particular point in time (e.g., scheduled operation, immediately upon request). In some embodiments, the remote user's schedule may be known. In some instances, the remote user's schedule may be pre-arranged. For example, the remote user may block out an hour if he or she knows that he or she will be busy supporting a particular procedure. The remote user's schedule may optionally be updated in real-time. For example, if a remote user takes a remote call, the vendor representative schedule may be automatically updated to indicate that the remote user is not available, until the remote user is done with the call. In some instances, if the remote user is off working hours, or is unable to work, the remote user may provide an input that may indicate that the remote user is unavailable. The medical resource intelligence system may be synchronized with a remote user's calendar (e.g., Outlook calendar, Google calendar, or any other type of calendar) and may automatically be able to pull in some of the scheduling information. In some embodiments, the remote user availability may be known and updated in real-time. If the remote user is not available or is not predicted to available at the time needed, then the remote user may be removed from the pool of remote users.

The vendor rep can provide a preference list of remote users (e.g., vendors) to be contacted if a preferred remote user is not available. The vendor rep can provide a preference list of remote users based on the location of the remote users, the availability of the remote users, the experience level of the remote users, or the preference list provided by the user. The preference list can be provided with any level of specificity or condition. For example, the preference list may be different for different medical practitioners. The preference list may be different for different medical products. The preference list may vary depending on one or more factors. The experience level of the remote users can be relative to a particular piece of equipment. The experience level of the remote users can be relative to a particular procedure. The experience level of the remote users can be relative to their experience with a particular user or location (e.g., hospital or operating facility). In some embodiments, a preference list provided by the vendor may be created based on the vendor's previous personal experience with other vendor representatives. A medical practitioner may prefer to rely on personalized recommendations provided by a trusted vendor representative. The vendor representative may be in a uniquely advantageous position to understand which colleague or other individual may best serve in a particular situation.

The preference list of remote users can be stored in a database of the vendor. The database can be a cloud-based database. The preference list may have any format (e.g., Word, Excel, HTML, etc.). The vendor representative may provide a ranking or order to preferred back-up vendor representatives. The practitioner may or may not be able to view or alter the preference list. In some instances, the preference list may be provided automatically and take priority over other factors when a system attempts to contact other vendor representatives. In some instances, the preference list may be weighed against other factors when a system attempts to contact other vendor representatives. In some instances the preference list can be ordered based on a user preference before availability of a vendor representative. In some instances the preference list can be ordered based on an availability of a vendor representative before an experience. In some instances, the preference list can be configured to weigh a user preference before an availability of a vendor representative. In some instances, the preference list can be configured to weigh an availability of a vendor representative before a user preference. In some instances, the preference list can be ordered based on a vendor association with a particular network or expertise.

In some embodiments, the machine learning may utilize deep convolution neural networks/Faster R-CNN Nast NasNet (COCO). The machine learning may utilize any type of convolutional neural network (CNN) and/or recurrent neural network (RNN). Shift invariant or space invariant neural networks (SIANN) may also be utilized. Image classification, object detection and object localization may be utilized. Any machine learning technique known or later developed in the art may be used. For instance, different types of neural networks may be used, such as Artificial Neural Net(ANN), Convolution Neural Net (CNN), Recurrent Neural Net (RNN), and/or their variants.

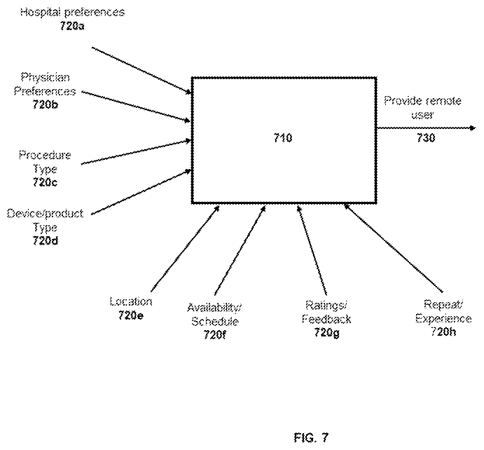

FIG. 7 shows an example of one or more factors that may be considered in identifying a remote user (e.g., a vendor representative, physician specialist etc.) to provide support in a particular instance, in accordance with embodiments of the invention. In some instances, it may be desirable to identify a remote user ahead of time, before the procedure. Alternatively, the remote user may be identified during the procedure. Any description herein of a remote user may apply to a physician specialist, consultant, medical professional, or vendor representative.

Various factors may be considered in identifying a remote user to provide support in a particular instance. Before or during a medical procedure, it may be desirable to identify a remote user to provide support. The medical console or other devices may be used to contact the remote user to provide support. The remote user support may be arranged before beginning the medical procedure. Alternatively, during the medical procedure, it may be determined that remote user support is needed, and an individual may be identified and contacted.

The various factors provided herein are provided by way of example only. Any other factors may be considered in the place of, or in addition to the factors provided herein. In some instances, not all of the factors illustrated herein are required. In some embodiments, 1, 2, 3, 4, 5, 6, 7, 8, 9, 10, 15, 20, 30, 50, or more of the factors provided herein are assessed in determining the identity of the remote user to provide support. Any number of factors, such as those more than any of the numbers provided, less than any of the numbers provided, or in between any of the numbers provided, may be considered.

A medical resource intelligence system 710 may assess any of the factors and provide a remote user identification 730 based on the factors.

An example of a factor that may be considered are health care facility preferences 720a. For examples, each hospital, clinic, care center, or any other type of health care facility may have preferred remote users. In some embodiments, a health care facility may have a pool of approved remote users. In some instances, health care facilities may have rankings or tiers (e.g., levels) of remote users that are preferred. This may be based on arrangements with the vendor, past experience with the remote users, recommendations, or any other factors.

Another example of a factor that may be considered are physician preferences 720b. For examples, each medical personnel may have preferred remote users. For example, a surgeon may have remote users he or she prefers from working with in the past. In some embodiments, a particular medical personnel may have a pool of approved or preferred remote users. In some instances, medical personnel may have rankings or tiers (e.g., levels) of remote users that are preferred. This may be based on arrangements with the remote user, past experience with the remote users, recommendations, product experience, procedure experience, specialty, or any other factors.

Any description herein of healthcare facility preferences or physician preferences may also apply to department or group preferences. For example some departments or groups may have preferences for remote users.

In some embodiments, physician preferences may depend on health care facility or group/department preferences. For example, if a health care facility only approves a particular pool of remote users, the medical personnel may have preferences from within the pool of remote users. In other examples, the health care facility may not be strict about the remote users, but may have a preferred list, which the individual medical personnel may or may not choose to defer to.

In another example, procedure type 720c may be a factor that is considered. For example, particular remote users may only support particular procedure types, or may have greater experience with certain procedure types. In some embodiments, the factor may be binary (e.g., vendor does or does not support a particular procedure type or the remote user has a specialty relevant to a subset of procedures). Alternatively, the factor may have a greater gradient (e.g., may depend on the degree of experience or specialized knowledge the remote user has with particular procedure types, may depend on a preference ranking or score provided by the remote user to support particular procedure types).

Furthermore, medical product/device type 720d may be a factor to be considered. For example, particular remote users may only support particular medical products, or may have greater experience with certain medical products. This may refer to categories of medical products (e.g., stents), or very particular medical products (e.g., stent model ABCD). In some embodiments, the factor may be binary (e.g., vendor does or does not support a particular medical product or product category). Alternatively, the factor may have a greater gradient (e.g., may depend on the degree of experience the remote user has with particular medical product or product category, may depend on a preference ranking or score provided by the remote user to support particular medical products or product categories).

Optionally, locations 720e may be considered as a factor. For examples, the locations of the various health care facilities may be known. The locations for the remote users (real-time location, address on file, office location, residential location, etc.) may be known. In some embodiments, the relative locations between the health care facilities and the remote users may be considered. In some embodiments, preferences may be given if they are closer together. In another example, preferences may be given for the same time zone or closer time zones. Alternatively, location may not be a factor, or may be a relatively weak factor when remote users are providing support remotely.

In some embodiments, availability and scheduling 720f may be considered when identifying a particular remote user to provide support. In some embodiments, the vendor support may be needed at a particular point in time (e.g., scheduled operation, immediately upon request). In some embodiments, the remote user schedule may be known. In some instances, the remote user schedule may be pre-arranged. For example, the vendor may block out an hour if he or she knows that he or she will be busy supporting a particular procedure. The remote user schedule may optionally be updated in real-time. For example, if a remote user takes a call, the remote user schedule may be automatically updated to indicate that the remote user is not available, until the remote user is done with the call. In some instances, if the remote user is off working hours, or is unable to work, the remote user may provide an input that may indicate that the remote user is unavailable. The medical resource intelligence system may be synchronized with a remote user's calendar (e.g., Outlook calendar, Google calendar, or any other type of calendar) and may automatically be able to pull in some of the scheduling information. In some embodiments, the remote user availability may be known and updated in real-time. If the remote user is not available or is not predicted to available at the time needed, then the remote user may be removed from the pool of remote users.

Similarly, if in the past, the remote user has shown him or herself to not be available when indicated as available, this may be a factor that may be considered. For example, if the remote users has had numerous missed calls, the remote user may be less likely to be identified as the recommended remote user.

Optionally, ratings or feedback about the various remote users 720g may be considered as a factor. For examples, personnel that have interacted with the remote users before may provide feedback or other types of ratings or scores. For example, when a session is completed, a medical personnel may be presented with an opportunity to rate the experience working with the remote user. Quantitative (e.g., score, rating) and/or qualitative feedback (e.g., comments) may be collected and/or considered. The feedback may be aggregated and/or accumulated, which may be a factor in determining whether the remote user is identified. In some embodiments, the higher ratings/feedback will result in the remote user more likely being selected.

In some embodiments, the entirety of the feedback/ratings may be accumulated and used when identifying the remote user. In other instances, the feedback/ratings may be considered within the context, such as other factors. For example, a remote user may have a low rating for a particular medical product, but may have a high rating for a different medical product (e.g., second product). If the support requested is for the second product, the remote user may be assessed on the higher rating. In another example, if the remote user has a high rating from the individuals at a first health care facility and a low rating from individuals at a second health care facility, the ratings may be considered in the context of the health care facility requesting the support.

A remote user's experience 720h may be considered as a factor when making the determination. For instance, the remote user's experience at a particular health care facility may be considered (e.g., number of times the remote user has provided support, the duration of the support provided, etc.). In some instances, the experience with particular individuals or groups/departments at a facility may be considered (e.g., a remote user may have worked extensively with a particular surgeon, etc.). The remote user's experience supporting a particular product (e.g., product category, product model) may be considered. Similarly, the remote user's experience supporting a particular procedure or procedure type may be considered. The experience may be considered in conjunction with ratings. In some instances it may be desirable to use a remote user who has a large degree of experience with the product, procedure, personnel, and/or location.

In some embodiments, the outcomes may be considered in conjunction with the remote user experience. For example, when a remote user has supported a particular procedure type, the procedure types may be assessed to determine whether outcomes differ (e.g. are more positive, more negative) based on remote user support. If a remote user supporting a particular procedure type tends to result in a more favorable outcome for that procedure type, it may be more desirable to use that remote user again. In another example, when a remote user has supported a particular product, the procedures using that product may be assessed to determine whether outcomes differ (e.g. are more positive, more negative) based on remote user support. If a remote user supporting a particular product tends to result in a less favorable outcome for that procedures using that product, it may be less desirable to use that remote user again. In some embodiments, outcome may depend on the success of the procedure and/or how the patient recovers after the procedure. In some instances, outcome may depend on the length and/or efficiency of the procedure as well. For example, if a procedure takes much longer to complete than anticipated, this may be a less favorable outcome than a procedure that ended as scheduled.

The medical resource intelligence system 710 may consider any of the factors provided above. In some embodiments, the factors may be weighted equally in identifying one or more remote users to support a particular procedure. Alternatively, different weights may be provided. For example, feedback/ratings may be rated higher than location. In some instances, one or more factors may be binary/prohibitive. For example, if a remote user is not available, the other factors may not matter, and then remote user may not be selected. In some instances, some factors may be given deference relative to other factors. For example, health care facility rules may trump individual physician preferences. The factors may be updated in real-time. The medical resource intelligence system may utilize machine learning to identify a remote user to support a procedure.

In some instances, the medical resource intelligence system may make a recommendation for a remote user based on the factors provided. The recommended remote user may be displayed to medical personnel or other individuals who may make the determination/confirmation whether to proceed with the recommended remote user. If the individual confirms the recommended remote user, then the remote user may be contacted and/or the schedule for the remote user may be updated for the procedure.

If the individual does not confirm the recommended remote user, the individual may be given the opportunity to select another remote user. The individual may directly enter the identity of the remote user the individual wishes to contact. In another example, a second recommended remote user option may be displayed to the individual, and so forth. The medical resource intelligence system may automatically rank recommended remote users, and start at the top of the list, and move down as needed. For example, if contact is requested with the first choice remote user, but the contact is not made, the system may move on to the second choice remote user.

In some instances, the medical resource intelligence system may automatically attempt to connect with the identified remote user. For example, once the first choice remote user has been identified, the system may automatically attempt to connect with the remote user, or update the schedule of the remote user for a current or future procedure. If the connection with the remote user is unsuccessful, the system may automatically move on to the next choice remote user and so forth. This may occur without requiring user intervention or input.

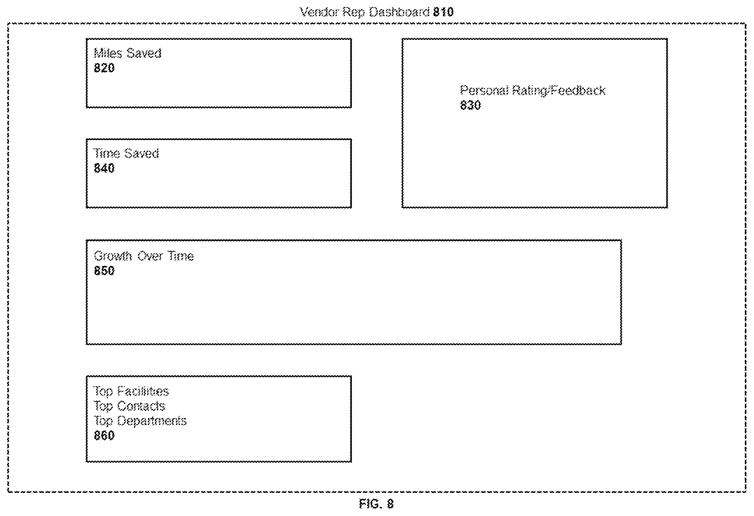

FIG. 8 shows an example of a user interface displaying medical resource intelligence to a remote user 810, in accordance with embodiments of the invention.

Any type of medical resource intelligence that may be useful to the remote user may be displayed on the user interface. The categories provided herein are provided by way of example only, and other types of medical resource intelligence data may be provided in any format. Furthermore, the arrangements provided herein are provided by way of example only, and alternative arrangements may be used. The placement and/or manner in displaying particular intelligence may be displayed. For example, they may be displayed as text, bar graphs, line graphs, pie charts, histograms, symbols, or any other arrangement.

Information about distance saved from travel by utilizing a remote system 820 may be provided for a remote user. For example, a remote user may provide support to one or more health care facility remotely. By not being required to travel to the health care facility, the remote user may save on distance traveled, which may also save time and allow the remote user to provide more support. The distance saved may be provided relative to any location (e.g., the remote user's home, the remote user's office, or any other reference location). The distance saved may be calculated in miles, kilometers, or any other unit of distance.

Information about time saved by utilizing a remote system 840 may be provided for a remote user. For example, a remote user may provide support to one or more health care facility remotely. By not being required to travel to the health care facility, and undergo possible physical processing and delay, the remote user may save time, which may allow the remote user to provide more support. The time saved may be calculated based on distance saved. In some instances, the time saved may depend on the amount of time that it would take to travel the distance. In some instances, map or traffic information may be utilized in order to determine the amount of time that was saved by not having to travel between particular locations at particular points in time. In some instances, information such as traffic data from the time of the procedure may be used to accurately calculate how much time was saved. Alternatively, more general information, such as speed limits, etc. may be utilized in calculating time saved by not traveling a particular distance. Other factors may be used to calculate time saved. For instance, the time to find parking and check in at a health care facility, other factors that may typically, add time required for a remote user to perform in-person support. The time saved may be calculated in weeks, days, hours, minutes, seconds, or any other unit of time.

A remote user may be able to view a personal rating and/or feedback 830. For example, after a remote user provides support for a procedure, associated medical personnel may be able to provide a rating and/or feedback. The rating may be a quantitative measure. For example, the remote user may receive a numerical score, such as a rating from 1 to 5, or between any other numbers. In another example, the remote users may receive a letter grade, such as A through E. A remote user may receive an aggregate rating or score based on the ratings supplied for the various procedures that remote user has provided. The score may be based on the entire history of ratings provided to the remote user. In other instances, the sore may be based on the ratings provided within a particular timeframe (e.g., within the past several years, within the past year, within the past quarter, within the past month, etc.). In some instances, a remote user may be able to view a score for a particular product that the remote user has provided support, a particular medical procedure, a particular health care facility, or particular medical personnel that the remote user has provided support. Any of this information may be indexed, and the medical personnel may be able to view a score in relation to any of these parameters, or others.

In some instances, the remote user may receive qualitative feedback. For example, medical personnel may leave comments about the remote user's performance. This may include positive and/or negative comments. The remote user may be able to view the comments in order to understand areas where the remote user could improve, as well as areas of strength.

A remote user growth over time 850 may be displayed to the remote user. For example, for a particular remote user, past amount of support (e.g., length of time providing support, number of medical procedures supported, revenue generated, etc.) may be displayed. Forecasted amount of support may or may not be displayed as well. The remote user support over time may be displayed.

The remote user may be able to view growth/support amount over time with any level of specificity. For example, the remote user may be able to view growth for a particular product that the remote user supports. The remote user may potentially support multiple products. In some embodiments, a user may toggle between views of growth (and/or forecast) for different products. In some instances, the views of growth (and/or forecast) may overlay one another and/or be viewed simultaneously.

In another example, the remote user may be able to view growth for a particular procedure type that the remote user supports. The remote user may potentially support multiple procedure types. In some embodiments, a user may toggle between views of growth (and/or forecast) for different procedure types. In some instances, the views of growth (and/or forecast) may overlay one another and/or be viewed simultaneously.

In a further example, the remote user may be able to view growth for a particular health care facility, department, or individual medical personnel that the remote user supports. The remote user may potentially support multiple health care facilities, departments, or medical personnel. In some embodiments, a user may toggle between views of growth (and/or forecast) for different health care facilities, departments and/or medical personnel. In some instances, the views of growth (and/or forecast) may overlay one another and/or be viewed simultaneously.

In some instances, information about top facilities, top contacts, and/or top departments 860 may be presented to remote user. For example, top facilities that use the remote user's services may be presented to the remote user. The top facilities may be calculated based on the number of products supported, the number of procedures supported, the length of time of support provided, or any other factor. The top facilities may be displayed so that they are ranked, with the facility that remote user provides the most support to is displayed at the top. This may advantageously permit a remote user to focus attention on facilities with which a lot of business is already conducted, which may be useful for purposes of allocating resources or making other decisions.

Similarly, top departments or top medical personnel that use the remote user's services may be presented to the remote user. The top departments and/or medical personnel may be calculated based on the number of products supported, the number of procedures supported, the length of time of support provided, or any other factor. The top departments or medical personnel may be displayed so that they are ranked, with the depat intent or medical personnel that remote user provides the most support to is displayed at the top. This may advantageously permit a remote user to focus attention on departments or medical personnel with which a lot of business is already conducted, which may be useful for purposes of allocating resources or making other decisions.

The schematic of the user interface is provided by way of example only. Any of the data described herein may be displayed in each of their own visually discernible regions. The regions may be provided adjacent to one another, or above or below one another. The regions may be displayed simultaneously on the same user interface. The regions may have a fixed location or variable location. The regions may have any size or shape. The regions may have any placement relative to one another and are not limited to those provided herein.

Computer Control Systems

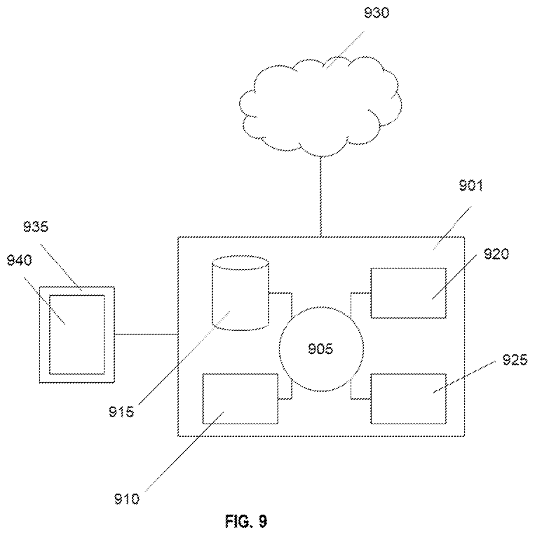

The present disclosure provides computer control systems that are programmed to implement methods of the disclosure. FIG. 9 shows a computer system 901 that is programmed or otherwise configured to facilitate communications between remote users and medical personnel that may need a remote user's support. The computer system may facilitate communications between a rep communication device and a local communication device. The computer system may automatically interface with one or more inventory systems or resource management systems of one or more health care facilities. The computer system may access information about one or more remote users, such as one or more remote users. The computer system may automatically determine one or more remote users that may be suitable for providing support for a medical procedure utilizing a medical product. The computer system may provide medical resource intelligence, such as information about past medical product usage and forecasting future usage. The computer system may provide remote user productivity measures. The computer system may advantageously provide engagement information. The computer system can be an electronic device of a user or a computer system that is remotely located with respect to the electronic device. The electronic device can be a mobile electronic device.

The computer system 901 may include a central processing unit (CPU, also “processor” and “computer processor” herein) 905, which can be a single core or multi core processor, or a plurality of processors for parallel processing. The computer system also includes memory or memory location 910 (e.g., random-access memory, read-only memory, flash memory), electronic storage unit 915 (e.g., hard disk), communication interface 920 (e.g., network adapter) for communicating with one or more other systems, and peripheral devices 925, such as cache, other memory, data storage and/or electronic display adapters. The memory 910, storage unit 915, interface 920 and peripheral devices 925 are in communication with the CPU 905 through a communication bus (solid lines), such as a motherboard. The storage unit 915 can be a data storage unit (or data repository) for storing data. The computer system 901 can be operatively coupled to a computer network (“network”) 930 with the aid of the communication interface 920. The network 930 can be the Internet, an internet and/or extranet, or an intranet and/or extranet that is in communication with the Internet.

The network 930 in some cases is a telecommunication and/or data network. The network can include one or more computer servers, which can enable distributed computing, such as cloud computing. For example, one or more computer servers may enable cloud computing over the network (“the cloud”) to perform various aspects of analysis, calculation, and generation of the present disclosure, such as, for example, capturing a configuration of one or more experimental environments; storing in a registry the experimental environments at each of one or more time points; performing one or more experimental executions which leverage experimental environments; providing outputs of experimental executions which leverage the environments; generating a plurality of linkages between the experimental environments and the experimental executions; and generating one or more execution states corresponding to the experimental environments at one or more time points. Such cloud computing may be provided by cloud computing platforms such as, for example, Amazon Web Services (AWS), Microsoft Azure, Google Cloud Platform, and IBM cloud. The network, in some cases with the aid of the computer system 901, can implement a peer-to-peer network, which may enable devices coupled to the computer system to behave as a client or a server.

The CPU 905 can execute a sequence of machine-readable instructions, which can be embodied in a program or software. The instructions may be stored in a memory location, such as the memory 910. The instructions can be directed to the CPU, which can subsequently program or otherwise configure the CPU to implement methods of the present disclosure. Examples of operations performed by the CPU can include fetch, decode, execute, and writeback.

The CPU 905 can be part of a circuit, such as an integrated circuit. One or more other components of the system can be included in the circuit. In some cases, the circuit is an application specific integrated circuit (ASIC).

The storage unit 915 can store files, such as drivers, libraries and saved programs. The storage unit can store user data, e.g., user preferences and user programs. The computer system 901 in some cases can include one or more additional data storage units that are external to the computer system, such as located on a remote server that is in communication with the computer system through an intranet or the Internet.

The computer system 901 can communicate with one or more remote computer systems through the network 930. For instance, the computer system can communicate with a remote computer system of a user (e.g., a user of an experimental environment). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system via the network.

Methods as described herein can be implemented by way of machine (e.g., computer processor) executable code stored on an electronic storage location of the computer system 901, such as, for example, on the memory 910 or electronic storage unit 915. The machine executable or machine readable code can be provided in the form of software. During use, the code can be executed by the processor 905. In some cases, the code can be retrieved from the storage unit and stored on the memory for ready access by the processor. In some situations, the electronic storage unit can be precluded, and machine-executable instructions are stored on memory.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 901, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 901 can include or be in communication with an electronic display 935 that comprises a user interface (UI) 940 for providing, for example, selection of an environment, a component of an environment, or a time point of an environment. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 905. The algorithm can, for example, capture a configuration of one or more experimental environments; store in a registry the experimental environments at each of one or more time points; perform one or more experimental executions which leverage experimental environments; provide outputs of experimental executions which leverage the environments; generate a plurality of linkages between the experimental environments and the experimental executions; and generate one or more execution states corresponding to the experimental environments at one or more time points.

It should be understood from the foregoing that, while particular implementations have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

1. A method of tracking medical product usage during a medical procedure, said method comprising:
(a) recognizing, with aid of one or more sensors, one or more medical products;
(b) identifying, with aid of the one or more sensors, a procedure associated with the one or more medical products and a remote user to contact; and
(c) communicating to an operator whether the remote user is available.

2. The method of claim 1, wherein recognizing comprises scanning one or more graphical codes on the one or more medical products.

3. The method of claim 2, wherein the one or more graphical codes comprise a barcode or QR code.

4. The method of claim 1 wherein identifying the first remote user is based on a preference list.

5. The method of claim 1, wherein identifying the remote user is based on a plurality of factors including availability, experience, location of the remote user, location of the operator, or identity of a healthcare facility.

6. The method of claim 1, wherein (c) comprises identifying a second remote user when the remote user is not available.

7. The method of claim 6, wherein the second remote user is identified based on a preference list of the operator.

8. The method of claim 4, wherein the second remote user is specified by the operator.

9. The method of claim 1, wherein (c) comprises accessing a schedule of the remote user.

10. The method of claim 9, wherein the schedule of the remote user is automatically updated.

11. The method of claim 1, further comprising, before (a), recognizing the operator.

12. The method of claim 11, wherein the operator is recognized via voice recognition, password login, facial identification, or any combination thereof.

13. The method of claim 1, further comprising (d) monitoring the length of time the operator is using the one or more medical products.

14. The method of claim 13, further comprising (e) determining an amount of time the one or more medical products is expected to be used based on the identified procedure.

15. The method of claim 14, further comprising, upon recognizing that the length of time the operator is using the one or more medical products is greater than the amount of the one or more medical products is expected to be used based on the identified procedure, prompting the operator with an option to contact the first remote user.

16. The method of claim 15, wherein commands from the operator are received via a touchless interface.

17. The method of claim 16, wherein the touchless interface comprises voice control or live video.

18. A method of tracking medical product usage during a medical procedure, said method comprising:
(a) recognizing, with aid of one or more sensors, one or more medical products;
(b) identifying, with aid of the one or more processors, a procedure associated with the one or more medical products and a remote user to contact; and
(c) automatically connecting the remote user to an operator.

19. The method of claim 18, further comprising in (b) monitoring the length of time the operator is using the one or more medical products.

20. The method of claim 18, further comprising in (b) determining an amount of time the one or more medical products is expected to be used based on the identified procedure

21.-22. (canceled)
